(19) United States
(12) Patent Application Publication
(10) Pub. No.: US 2025/0262008 A1
(43) Pub. Date: Aug. 21, 2025

(54) SYSTEM AND METHOD FOR POSITIONING A ROBOTIC ARM AND A SURGICAL ROBOT STATION FOR SURGERY

(71) Applicant: GLOBUS MEDICAL, INC., AUDUBON, PA (US)

(72) Inventors: Olivier Chappuis, Lutry (CH); Szymon Kostrzewski, Lausanne (CH); Jaroslaw Glowacki, Preverenges (CH); Daniel Gehriger, Lausanne (CH); Benoit Brot, Lausanne (CH); Marton Antal, Lausanne (CH)

(21) Appl. No.: 19/053,691

(22) Filed: Feb. 14, 2025

Related U.S. Application Data
(60) Provisional application No. 63/553,833, filed on Feb. 15, 2024.

(51) Int. Cl. A61B 34/30; A61B 34/20; A61B 90/00; A61F 2/46

(52) U.S. Cl. CPC A61B 34/30; A61B 34/20; A61B 90/06; A61B 90/361; A61F 2/461; A61B 2034/2057; A61B 2034/305; A61B 2090/067; A61B 2090/365

(57) ABSTRACT

A surgical robot system can determine a plurality of actions to be completed by a surgical robot station during a surgery. The surgical robot system can determine potential positions in an operating room that the surgical robot station can be positioned during the surgery. The surgical robot system generates a score associated with the determined positions and determines an optimal position of the surgical robot station for display based on the generated scores.

### CROSS REFERENCE TO RELATED APPLICATIONS

This application claims the benefit of priority to U.S. Provisional Application No. 63/553,833, filed Feb. 15, 2024, the contents of which are incorporated by reference herein in their entirety.

### TECHNICAL FIELD

The present disclosure relates to medical devices and systems, and more particularly to determining a desired position of a surgical robot station for a surgery, guiding the surgical robot station to the desired position, and pathing a robotic arm of the surgical robot station

### BACKGROUND

Patient satisfaction with the outcome of a surgery can depend upon the surgeon's expertise with best practices and use of rapidly emerging innovations in surgical procedures including new and customized implant designs, computer-assisted navigation, and surgical robot systems.

There are a number of surgical interventions requiring osteotomy, i.e. cutting an anatomical structure such as a bone along a target plane. Total knee arthroplasty typically requires cutting both the femoral epiphysis and tibial epiphysis in order to remove the damaged bone and cartilage and install a knee prosthesis. A surgeon may perform five or more cuts on the femur and one or more cuts on the tibia using an oscillating surgical saw.

During orthopedic surgeries, including joints and knees, it is important to accurately align and stabilize the saw while cutting a desired location on a bone. The surgeon's limited visibility to the surgical site combined with the difficultly in controlling movement of the saw creates a risk that an undesired part of a bone or adjacent tissue becomes cut. Vibrations generated by the saw while cutting can reduce the accuracy of the cuts. During knee surgery, the precision of a bone cut (planar cuts) affects how precisely the implant can be connected to the exposed bone.

In some conventional systems, a direct sagittal saw blade guidance structure may use a passive kinematics positioned in the space by a robotic arm, which to constrain the blade in its resection plane. The passive structure, further designated as an end effector arm, may include a three linkage serial structure offering 3 degrees of freedom (two translations and one rotation) to the blade. The robotic arm may have a limited range requiring the guidance structure to be positioned at different positions relative to the operating table based on an anatomy of the patient.

### SUMMARY

According to some embodiments, a surgical robot system is provided. The surgical robot system includes processing circuitry and memory coupled to the processing circuitry. The memory has instructions stored therein that are executable by the processing circuitry to cause the surgical robot system to perform operations. The operations cause the robot system to determine a plurality of actions to be completed by a surgical robot station during a surgery. The operations further cause the robot system to determine a plurality of potential positions in an operating room that the surgical robot station can be positioned during the surgery. The operations further cause the robot system to generate a score associated with a potential position of the plurality of potential positions based on estimated movement of the surgical robot station required to perform the plurality of actions during the surgery from the potential position.

According to other embodiments, a surgical robot system is provided. The surgical robot system includes processing circuitry and memory coupled to the processing circuitry. The memory has instructions stored therein that are executable by the processing circuitry to cause the surgical robot system to perform operations. The operations cause the robot system to receive an indication of a position of each object of a first plurality of objects in a surgical environment from a camera tracking system. The operations further cause the robot system to receive an indication of a starting position of a robotic arm of a surgical robot station from the camera tracking system. The operations further cause the robot system to determine a target position of the robotic arm based on a surgery being performed by the surgical robot station. The operations further cause the robot system to determine a path for the robotic arm to move from the starting position to the target position based on the starting position, the target position, and the position of each object of the first plurality of objects. The operations further cause the robot system to, subsequent to the robotic arm beginning to move along the path and prior to the robotic arm arriving at the target position, receive an indication of a current position of a second plurality of objects in the surgical environment from the camera tracking system. The operations further cause the robot system to, subsequent to the robotic arm beginning to move along the path and prior to the robotic arm arriving at the target position, receive an indication of a current position of the robotic arm from the camera tracking system. The operations further cause the robot system to update the path for the robotic arm based on the current position of the robotic arm and the current position of the second plurality of objects.

According to other embodiments, a surgical robot system, a method, a surgical device, a computer program, a computer program product, or a non-transitory computer readable medium is provided to perform one or more of the operations described above. It is intended that all such surgical robot systems, surgical devices, computer programs, computer program products, and non-transitory computer readable mediums be included within this description, be within the scope of the present disclosure, and be protected by the accompanying claims. Moreover, it is intended that all embodiments disclosed herein can be implemented separately or combined in any way and/or combination.

Some embodiments herein provide one or more technical advantages. In some embodiments, determining an optimal position of a surgical robot system can reduce a number of times that the surgical robot system must be moved during a surgery. In some examples, reducing the number of times that surgical robot system must be moved can reduce the amount of time required to complete the surgery, reduce the risk of complications during the surgery, and improve the patient satisfaction with an outcome of the surgery.

In additional or alternative embodiments, guiding placement of the surgical robot system at the optimal position can improve the stability of a robotic arm of the surgical robot system during a surgery. In some examples, improving the stability of the robotic arm can improve a precision and/or an accuracy of a surgical action performed by (or assisted by) the surgical robot system.

In additional or alternative embodiments, dynamically updating a path of a robotic arm from a first position to a second position can prevent collisions between the robotic arm and other objects within the surgical environment. In some examples, preventing collisions can reduce the amount of time required to complete the surgery, reduce the risk of complications during the surgery, and improve the patient satisfaction with an outcome of the surgery.

In additional or alternative embodiments, dynamically updating a path of a robotic arm from a first position to a second position can improve an accuracy and/or precision of the positioning of the robotic arm at the second position. In additional or additional or alternative examples, improving an accuracy and/or precision of the positioning of the robotic arm at the second position can improve a precision and/or an accuracy of a surgical action performed by (or assisted by) the surgical robot system.

### BRIEF DESCRIPTION OF THE DRAWINGS

The accompanying drawings, which are included to provide a further understanding of the disclosure and are incorporated in a constitute a part of this application, illustrate certain non- limiting embodiments of inventive concepts. In the drawings:

FIG. 1 illustrates an embodiment of a surgical system according to some embodiments of the present disclosure;

FIG. 2 illustrates a surgical robot component of the surgical system of FIG. 1 according to some embodiments of the present disclosure;

FIG. 3 illustrates a camera tracking system component of the surgical system of FIG. 1 according to some embodiments of the present disclosure;

FIG. 4 illustrates an embodiment of a passive end effector that is connectable to a robot arm and configured according to some embodiments of the present disclosure;

FIG. 5 illustrates a medical operation in which a surgical robot and a camera system are disposed around a patient;

FIG. 6 illustrates an embodiment of an end effector coupler of a robot arm configured for connection to a passive end effector according to some embodiments of the present disclosure;

FIG. 7 illustrates an embodiment of a cut away of the end effector coupler of FIG. 6;

FIG. 8 illustrates a block diagram of components of a surgical system according to some embodiments of the present disclosure;

FIG. 9 illustrates a block diagram of a surgical system computer platform that includes a surgical planning computer which may be separate from and operationally connected to a surgical robot or at least partially incorporated therein according to some embodiments of the present disclosure;

FIG. 10 illustrates an embodiment of a C-Arm imaging device that can be used in combination with the surgical robot and passive end effector in accordance with some embodiments of the present disclosure;

FIG. 11 illustrates an embodiment of an O-Arm imaging device that can be used in combination with the surgical robot and passive end effector in accordance with some embodiments of the present disclosure; and

FIG. 12 illustrates an embodiment of a passive end effector configured in accordance with some embodiments of the present disclosure.

FIG. 13 is a schematic diagram illustrating an example of an overhead view of a surgical robot system arranged during a surgical procedure in a surgical room in accordance with some embodiments;

FIGS. 14-15 are schematic diagrams illustrating an example of a graphical interface displaying a current position of a surgical robot station relative to a desired position of the surgical robot station in accordance with some embodiments of the present disclosure;

FIG. 16 is a schematic diagram illustrating an example of a graphical interface displaying an indication that the surgical robot station is positioned at the desired position in accordance with some embodiments of the present disclosure;

FIGS. 17A-D are schematic diagrams illustrating an example of a stages of a robotic arm along a path from a starting position to a target position in accordance with some embodiments of the present disclosure;

FIG. 18 is a time lapsed illustration of a transition between a tibial resection and a femoral distal resection, which is the transition exhibiting the largest rotation of the robotic arm according an aspect of the present invention.

FIG. 19 is a flow chart illustrating an example of operations performed by a surgical robot system to position a surgical robot station in accordance with some embodiments of the present disclosure; and

FIG. 20 is a flow chart illustrating an example of operations performed by a surgical robot system to dynamically update a path for movement of a robotic arm in accordance with some embodiments of the present disclosure.

### DETAILED DESCRIPTION

Inventive concepts will now be described more fully hereinafter with reference to the accompanying drawings, in which examples of embodiments of inventive concepts are shown. Inventive concepts may, however, be embodied in many different forms and should not be construed as limited to the embodiments set forth herein. Rather, these embodiments are provided so that this disclosure will be thorough and complete, and will fully convey the scope of various present inventive concepts to those skilled in the art. It should also be noted that these embodiments are not mutually exclusive. Components from one embodiment may be tacitly assumed to be present or used in another embodiment.

A surgical robot system can be used in various surgeries to, for example, improve a precision of a surgical action and reduce an amount of time required to perform the surgical action. For example, some surgical robot systems include a base for securely positioning the surgical robot system to a position relative to a patient and a robotic arm that moves in three-dimension space (e.g., by bending, rotating, and extending) to position a surgical tool relative to the patient. During some surgeries, a surgical robot system can be used to perform and/or assist multiple surgical actions. Depending on the type of surgery and anatomy of the patient, the robotic arm of the surgical robot system may need to move the surgical tool between different positions and/or poses relative to the patient between different surgical actions. In some examples, the base of the surgical robot system may have to be moved during the surgery to allow the robotic arm to move the surgical tool to the correct position and/or pose relative to the patient. However, moving the surgical robot system during the surgery can be time consuming and increase the risk of complications.

Various embodiments herein describe a procedure to guide a user to optimally position and orient a surgical robot system around an operating table with a tracked patient positioned on it. In some examples, the optimal position may be a position that ensures that a surgeon can perform the surgery with the surgical robot system without the need for repositioning of the surgical robot system in the middle of the surgery. In additional or alternative examples, the optimal position may be a position that minimizes the number of times the surgical robot system must be repositioned in the middle of the surgery. In additional or alternative examples, the optimal position may be a position that allows a robotic arm of the surgical robot system to most easily, reliably, and/or stably reach each of a plurality of positions and/or poses relative to the patient.

As stated above, a robotic arm of a surgical robot system may need to move the surgical tool between different positions and/or poses relative to the patient between different surgical actions. A path can be determined for moving the robotic arm (and surgical tool) from a first position and/or pose to a second position and/or pose. However, during the time it takes for the robotic arm to move from the first position to the second position, objects in the environment of the robotic arm can move and risk collision with the robotic arm if it remains on the path. Furthermore, as these objects and the robotic arm move, they may move closer, farther, or out of sight of a camera tracking system, which can alter an accuracy of estimates of their current position relative to each other.

Various embodiments herein may additionally or alternatively describe dynamically updating the path of the robotic arm as it moves from a first position to a second position. In some examples, the path is updated periodically. In additional or alternative examples, the path is updated in response to a signal to stop movement of the robotic arm (e.g., detection of a collision or in response to a surgeon removing an input signal required for the robotic arm to move. In some embodiments, the path is updated based on new information regarding a position and/or mobility of the robotic arm or other objects in the surgical environment. In some examples, a proximity tolerance associated with an object in the surgical environment can change based on a speed and or frequency of movement of the object.

Although embodiments herein may be described in regards to a particular surgical robot system (e.g., an ExcelsiusFlex (“EFlex”)—Total Knee Arthroplasty (“TKA”) system), the innovations described herein can be applied to any suitable surgical robot system. Similarly, while embodiments herein may be described in regards to a particular surgical robot system (e.g., an EFlex station), the innovations described herein can be applied to any suitable surgical robot station.

In some embodiments herein, the terms “position,” “orientation,” and “pose” can be used interchangeably to refer to both a location and alignment of an object. In some examples, the term “pose” refers to the location (e.g., along 3 orthogonal axes) and/or the rotation angle (e.g., about the 3 orthogonal axes).

In some embodiments herein the terms robotic arm can refer to an end effector arm and/or a robotic arm that holds the end effector arm.

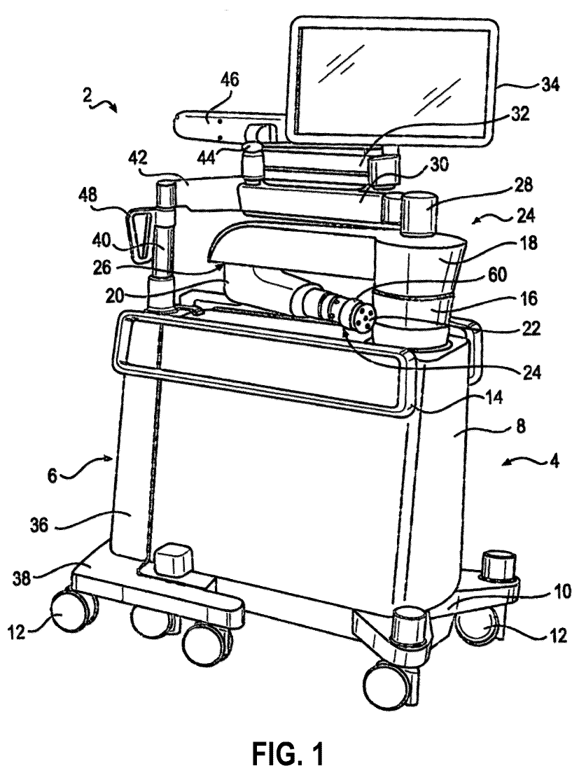

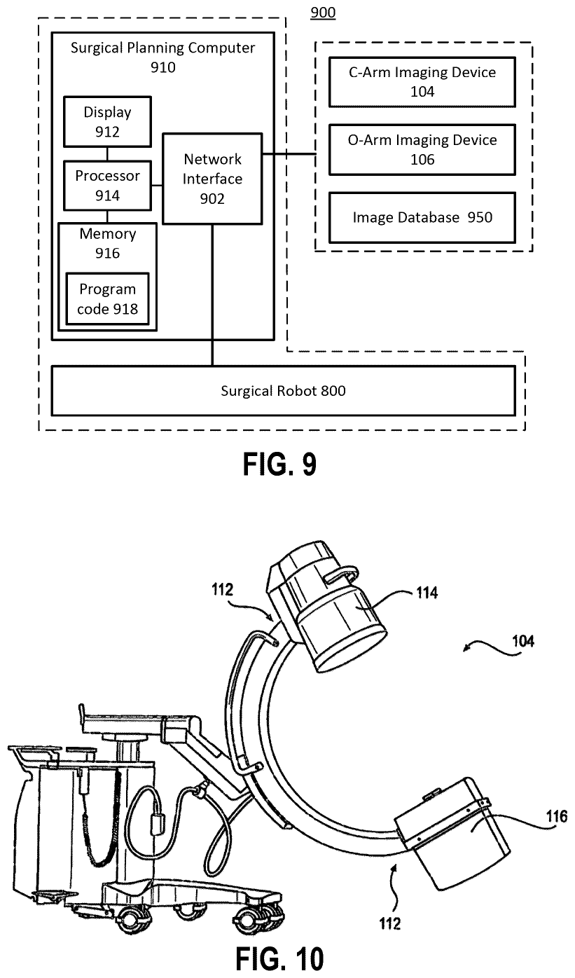

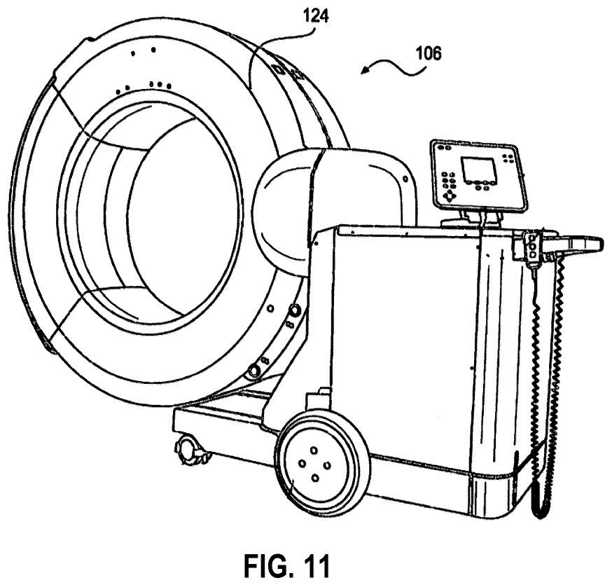

FIG. 1 illustrates an embodiment of a surgical system 2 according to some embodiments of the present disclosure. Prior to performance of an orthopedic surgical procedure, a three-dimensional (“3D”) image scan may be taken of a planned surgical area of a patient using, e.g., the C-Arm imaging device 104 of FIG. 10 or O-Arm imaging device 106 of FIG. 11, or from another medical imaging device such as a computed tomography (CT) image or Mill. This scan can be taken pre-operatively (e.g. few weeks before procedure, most common) or intra-operatively. However, any known 3D or 2D image scan may be used in accordance with various embodiments of the surgical system 2. The image scan is sent to a computer platform in communication with the surgical system 2, such as the surgical system computer platform 900 of FIG. 9 which includes the surgical robot 800 (e.g., the robot of surgical system 2 in FIG. 1) and a surgical planning computer 910. A surgeon reviewing the image scan(s) on a display device of the surgical planning computer 910 (FIG. 9) generates a surgical plan defining a target plane where an anatomical structure of the patient is to be cut. This plane is a function of patient anatomy constraints, selected implant and its size. In some embodiments, the surgical plan defining the target plane is planned on the 3D image scan displayed on a display device.

The surgical system 2 of FIG. 1 can assist surgeons during medical procedures by, for example, holding tools, aligning tools, using tools, guiding tools, and/or positioning tools for use. In some embodiments, surgical system 2 includes a surgical robot 4 and a camera tracking system 6. Both systems may be mechanically coupled together by any various mechanisms. Suitable mechanisms can include, but are not limited to, mechanical latches, ties, clamps, or buttresses, or magnetic or magnetized surfaces. The ability to mechanically couple surgical robot 4 and camera tracking system 6 can allow for surgical system 2 to maneuver and move as a single unit, and allow surgical system 2 to have a small footprint in an area, allow easier movement through narrow passages and around turns, and allow storage within a smaller area.

An orthopedic surgical procedure may begin with the surgical system 2 moving from medical storage to a medical procedure room. The surgical system 2 may be maneuvered through doorways, halls, and elevators to reach a medical procedure room. Within the room, the surgical system 2 may be physically separated into two separate and distinct systems, the surgical robot 4 and the camera tracking system 6. Surgical robot 4 may be positioned adjacent the patient at any suitable location to properly assist medical personnel. Camera tracking system 6 may be positioned at the base of the patient, at patient shoulders or any other location suitable to track the present pose and movement of the pose of tracks portions of the surgical robot 4 and the patient. Surgical robot 4 and camera tracking system 6 may be powered by an onboard power source and/or plugged into an external wall outlet.

Surgical robot 4 may be used to assist a surgeon by holding and/or using tools during a medical procedure. To properly utilize and hold tools, surgical robot 4 may rely on a plurality of motors, computers, and/or actuators to function properly. Illustrated in FIG. 1, robot body 8 may act as the structure in which the plurality of motors, computers, and/or actuators may be secured within surgical robot 4. Robot body 8 may also provide support for robot telescoping support arm 16. In some embodiments, robot body 8 may be made of any suitable material. Suitable material may be, but is not limited to, metal such as titanium, aluminum, or stainless steel, carbon fiber, fiberglass, or heavy-duty plastic. The size of robot body 8 may provide a solid platform supporting attached components, and may house, conceal, and protect the plurality of motors, computers, and/or actuators that may operate attached components.

Robot base 10 may act as a lower support for surgical robot 4. In some embodiments, robot base 10 may support robot body 8 and may attach robot body 8 to a plurality of powered wheels 12. This attachment to wheels may allow robot body 8 to move in space efficiently. Robot base 10 may run the length and width of robot body 8. Robot base 10 may be about two inches to about 10 inches tall. Robot base 10 may be made of any suitable material. Suitable material may be, but is not limited to, metal such as titanium, aluminum, or stainless steel, carbon fiber, fiberglass, or heavy-duty plastic or resin. Robot base 10 may cover, protect, and support powered wheels 12.

In some embodiments, as illustrated in FIG. 1, at least one powered wheel 12 may be attached to robot base 10. Powered wheels 12 may attach to robot base 10 at any location. Each individual powered wheel 12 may rotate about a vertical axis in any direction. A motor may be disposed above, within, or adjacent to powered wheel 12. This motor may allow for surgical system 2 to maneuver into any location and stabilize and/or level surgical system 2. A rod, located within or adjacent to powered wheel 12, may be pressed into a surface by the motor. The rod, not pictured, may be made of any suitable metal to lift surgical system 2. Suitable metal may be, but is not limited to, stainless steel, aluminum, or titanium. Additionally, the rod may comprise at the contact-surface-side end a buffer, not pictured, which may prevent the rod from slipping and/or create a suitable contact surface. The material may be any suitable material to act as a buffer. Suitable material may be, but is not limited to, a plastic, neoprene, rubber, or textured metal. The rod may lift powered wheel 12, which may lift surgical system 2, to any height required to level or otherwise fix the orientation of the surgical system 2 in relation to a patient. The weight of surgical system 2, supported through small contact areas by the rod on each wheel, prevents surgical system 2 from moving during a medical procedure. This rigid positioning may prevent objects and/or people from moving surgical system 2 by accident.

Moving surgical system 2 may be facilitated using robot railing 14. Robot railing 14 provides a person with the ability to move surgical system 2 without grasping robot body 8. As illustrated in FIG. 1, robot railing 14 may run the length of robot body 8, shorter than robot body 8, and/or may run longer the length of robot body 8. Robot railing 14 may be made of any suitable material. Suitable material may be, but is not limited to, metal such as titanium, aluminum, or stainless steel, carbon fiber, fiberglass, or heavy-duty plastic. Robot railing 14 may further provide protection to robot body 8, preventing objects and or personnel from touching, hitting, or bumping into robot body 8.

Robot body 8 may provide support for a Selective Compliance Articulated Robot Arm, hereafter referred to as a “SCARA.” A SCARA 24 may be beneficial to use within the surgical system 2 due to the repeatability and compactness of the robotic arm. The compactness of a SCARA may provide additional space within a medical procedure, which may allow medical professionals to perform medical procedures free of excess clutter and confining areas. SCARA 24 may comprise robot telescoping support 16, robot support arm 18, and/or robot arm 20. Robot telescoping support 16 may be disposed along robot body 8. As illustrated in FIG. 1, robot telescoping support 16 may provide support for the SCARA 24 and display 34. In some embodiments, robot telescoping support 16 may extend and contract in a vertical direction. Robot telescoping support 16 may be made of any suitable material. Suitable material may be, but is not limited to, metal such as titanium or stainless steel, carbon fiber, fiberglass, or heavy-duty plastic. The body of robot telescoping support 16 may be any width and/or height in which to support the stress and weight placed upon it.

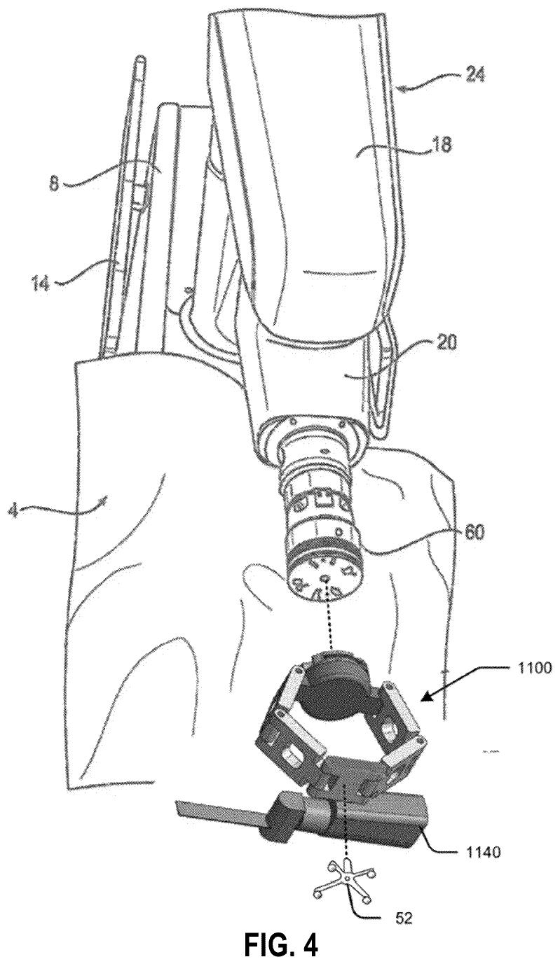

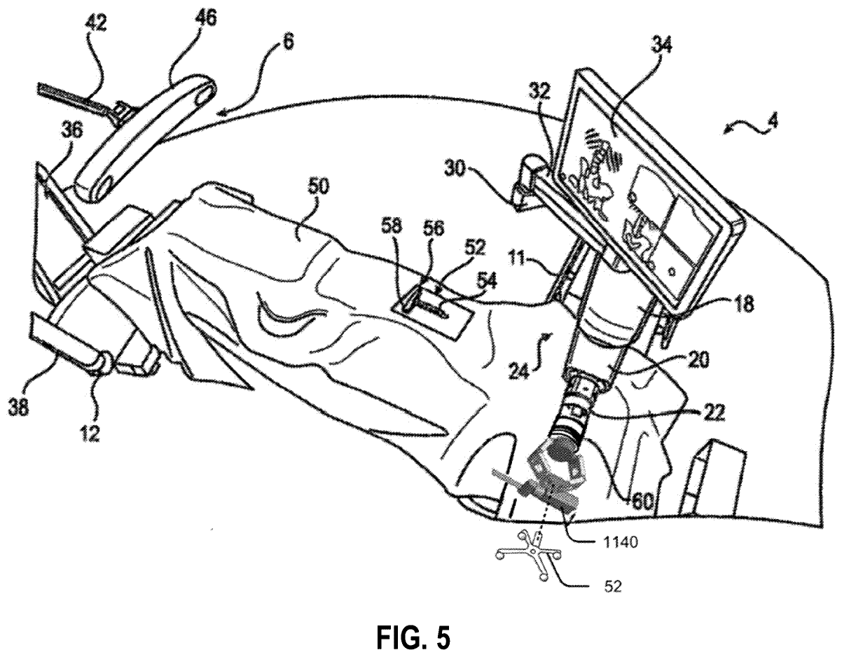

In some embodiments, medical personnel may move SCARA 24 through a command submitted by the medical personnel. The command may originate from input received on display 34 and/or a tablet. The command may come from the depression of a switch and/or the depression of a plurality of switches. Best illustrated in FIGS. 4 and 5, an activation assembly 60 may include a switch and/or a plurality of switches. The activation assembly 60 may be operable to transmit a move command to the SCARA 24 allowing an operator to manually manipulate the SCARA 24. When the switch, or plurality of switches, is depressed the medical personnel may have the ability to move SCARA 24 easily. Additionally, when the SCARA 24 is not receiving a command to move, the SCARA 24 may lock in place to prevent accidental movement by personnel and/or other objects. By locking in place, the SCARA 24 provides a solid platform upon which a passive end effector 1100 and connected surgical saw 1140, shown in FIGS. 4 and 5, are ready for use in a medical operation.

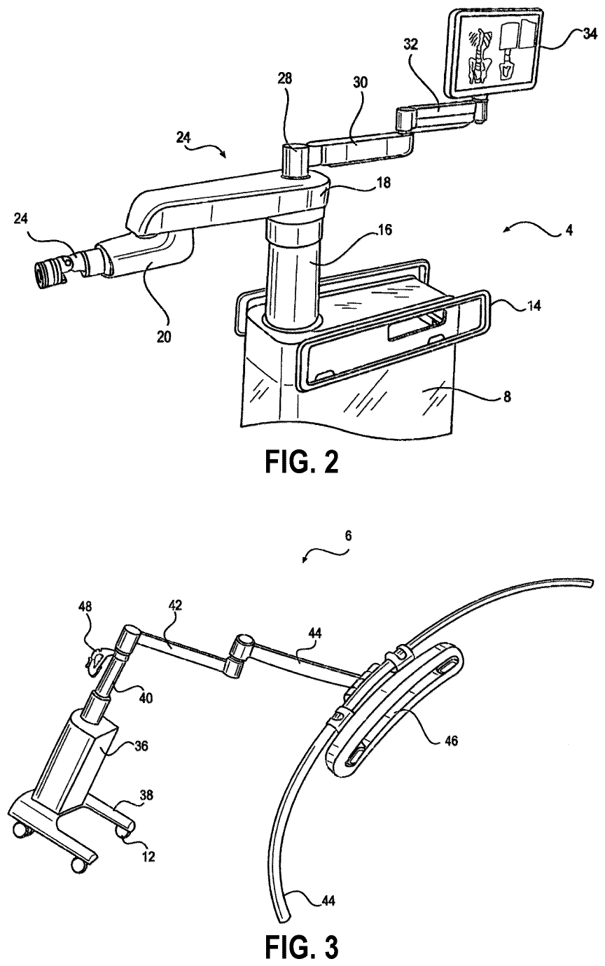

Robot support arm 18 may be disposed on robot telescoping support 16 by various mechanisms. In some embodiments, best seen in FIGS. 1 and 2, robot support arm 18 rotates in any direction in regard to robot telescoping support 16. Robot support arm 18 may rotate three hundred and sixty degrees around robot telescoping support 16. Robot arm 20 may connect to robot support arm 18 at any suitable location. Robot arm 20 may attach to robot support arm 16 by various mechanisms. Suitable mechanisms may be, but is not limited to, nuts and bolts, ball and socket fitting, press fitting, weld, adhesion, screws, rivets, clamps, latches, and/or any combination thereof. Robot arm 20 may rotate in any direction in regards to robot support arm 18, in embodiments, robot arm 20 may rotate three hundred and sixty degrees in regards to robot support arm 18. This free rotation may allow an operator to position robot arm 20 as planned.

The passive end effector 1100 in FIGS. 4 and 5 may attach to robot arm 20 in any suitable location. As will be explained in further detail below, the passive end effector 1100 includes a base, a first mechanism, and a second mechanism. The base is configured to attach to an end effector coupler 22 of the robot arm 20 positioned by the surgical robot 4. Various mechanisms by which the base can attach to the end effector coupler 22 can include, but are not limited to, latch, clamp, nuts and bolts, ball and socket fitting, press fitting, weld, adhesion, screws, rivets, and/or any combination thereof. The first mechanism extends between a rotatable connection to the base and a rotatable connection to a tool attachment mechanism. The second mechanism extends between a rotatable connection to the base and a rotatable connection to the tool attachment mechanism. The first and second mechanisms pivot about the rotatable connections, and may be configured to constrain movement of the tool attachment mechanism to a range of movement within a working plane. The rotatable connections may be pivot joints allowing 1 degree-of-freedom (DOF) motion, universal joints allowing 2 DOF motions, or ball joints allowing 3 DOF motions. The tool attachment mechanism is configured to connect to a surgical saw 1140 having a saw blade or saw blade directly. The surgical saw 1140 may be configured to oscillate the saw blade for cutting. The first and second mechanisms may be configured to constrain a cutting plane of the saw blade to be parallel to the working plane. Pivot joints may be preferably used for connecting the planar mechanisms when the passive end effector is to be configured to constrain motion of the saw blade to the cutting plane.

The tool attachment mechanism may connect to the surgical saw 1140 or saw blade through various mechanisms that can include, but are not limited to, a screw, nut and bolt, clamp, latch, tie, press fit, or magnet. In some embodiments, a dynamic reference array 52 is attached to the passive end effector 1100, e.g., to the tool attachment mechanism, and/or is attached to the surgical saw 1140. Dynamic reference arrays, also referred to as “DRAs” herein, are rigid bodies which may be disposed on a patient, the surgical robot, the passive end effector, and/or the surgical saw in a navigated surgical procedure. The camera tracking system 6 or other 3D localization system is configured to track in real-time the pose (e.g., positions and rotational orientations) of tracking markers of the DRA. The tracking markers may include the illustrated arrangement of balls or other optical markers. This tracking of 3D coordinates of tracking markers can allow the surgical system 2 to determine the pose of the DRA 52 in any space in relation to the target anatomical structure of the patient 50 in FIG. 5.

As illustrated in FIG. 1, a light indicator 28 may be positioned on top of the SCARA 24. Light indicator 28 may illuminate as any type of light to indicate “conditions” in which surgical system 2 is currently operating. For example, the illumination of green may indicate that all systems are normal. Illuminating red may indicate that surgical system 2 is not operating normally. A pulsating light may mean surgical system 2 is performing a function. Combinations of light and pulsation may create a nearly limitless amount of combinations in which to communicate the current operating conditions, states, or other operational indications. In some embodiments, the light may be produced by LED bulbs, which may form a ring around light indicator 28. Light indicator 28 may comprise a fully permeable material that may let light shine through the entirety of light indicator 28.

Light indicator 28 may be attached to lower display support 30. Lower display support 30, as illustrated in FIG. 2 may allow an operator to maneuver display 34 to any suitable location. Lower display support 30 may attach to light indicator 28 by any suitable mechanism. In embodiments, lower display support 30 may rotate about light indicator 28. In embodiments, lower display support 30 may attach rigidly to light indicator 28. Light indicator 28 may then rotate three hundred and sixty degrees about robot support arm 18. Lower display support 30 may be of any suitable length, a suitable length may be about eight inches to about thirty four inches. Lower display support 30 may act as a base for upper display support 32.

Upper display support 32 may attach to lower display support 30 by any suitable mechanism. Upper display support 32 may be of any suitable length, a suitable length may be about eight inches to about thirty four inches. In embodiments, as illustrated in FIG. 1, upper display support 32 may allow display 34 to rotate three hundred and sixty degrees in relation to upper display support 32. Likewise, upper display support 32 may rotate three hundred and sixty degrees in relation to lower display support 30.

Display 34 may be any device which may be supported by upper display support 32. In embodiments, as illustrated in FIG. 2, display 34 may produce color and/or black and white images. The width of display 34 may be about eight inches to about thirty inches wide. The height of display 34 may be about six inches to about twenty two inches tall. The depth of display 34 may be about one-half inch to about four inches.

In embodiments, a tablet may be used in conjunction with display 34 and/or without display 34. In embodiments, the table may be disposed on upper display support 32, in place of display 34, and may be removable from upper display support 32 during a medical operation. In addition the tablet may communicate with display 34. The tablet may be able to connect to surgical robot 4 by any suitable wireless and/or wired connection. In some embodiments, the tablet may be able to program and/or control surgical system 2 during a medical operation. When controlling surgical system 2 with the tablet, all input and output commands may be duplicated on display 34. The use of a tablet may allow an operator to manipulate surgical robot 4 without having to move around patient 50 and/or to surgical robot 4.

As illustrated in FIG. 5, camera tracking system 6 works in conjunction with surgical robot 4 through wired or wireless communication networks. Referring to FIGS. 1 and 5, camera tracking system 6 can include some similar components to the surgical robot 4. For example, camera body 36 may provide the functionality found in robot body 8. Robot body 8 may provide the structure upon which camera 46 is mounted. The structure within robot body 8 may also provide support for the electronics, communication devices, and power supplies used to operate camera tracking system 6. Camera body 36 may be made of the same material as robot body 8. Camera tracking system 6 may communicate directly to the tablet and/or display 34 by a wireless and/or wired network to enable the tablet and/or display 34 to control the functions of camera tracking system 6.

Camera body 36 is supported by camera base 38. Camera base 38 may function as robot base 10. In the embodiment of FIG. 1, camera base 38 may be wider than robot base 10. The width of camera base 38 may allow for camera tracking system 6 to connect with surgical robot 4. As illustrated in FIG. 1, the width of camera base 38 may be large enough to fit outside robot base 10. When camera tracking system 6 and surgical robot 4 are connected, the additional width of camera base 38 may allow surgical system 2 additional maneuverability and support for surgical system 2.

As with robot base 10, a plurality of powered wheels 12 may attach to camera base 38. Powered wheel 12 may allow camera tracking system 6 to stabilize and level or set fixed orientation in regards to patient 50, similar to the operation of robot base 10 and powered wheels 12. This stabilization may prevent camera tracking system 6 from moving during a medical procedure and may keep camera 46 from losing track of one or more DRAs 52 connected to an anatomical structure 54 and/or tool 58 within a designated area 56 as shown in FIG. 5. This stability and maintenance of tracking enhances the ability of surgical robot 4 to operate effectively with camera tracking system 6. Additionally, the wide camera base 38 may provide additional support to camera tracking system 6. Specifically, a wide camera base 38 may prevent camera tracking system 6 from tipping over when camera 46 is disposed over a patient, as illustrated in FIG. 5. Without the wide camera base 38, the outstretched camera 46 may unbalance camera tracking system 6, which may result in camera tracking system 6 falling over.

Camera telescoping support 40 may support camera 46. In embodiments, telescoping support 40 may move camera 46 higher or lower in the vertical direction. Telescoping support 40 may be made of any suitable material in which to support camera 46. Suitable material may be, but is not limited to, metal such as titanium, aluminum, or stainless steel, carbon fiber, fiberglass, or heavy-duty plastic. Camera handle 48 may be attached to camera telescoping support 40 at any suitable location. Cameral handle 48 may be any suitable handle configuration. A suitable configuration may be, but is not limited to, a bar, circular, triangular, square, and/or any combination thereof. As illustrated in FIG. 1, camera handle 48 may be triangular, allowing an operator to move camera tracking system 6 into a planned position before a medical operation. In embodiments, camera handle 48 may be used to lower and raise camera telescoping support 40. Camera handle 48 may perform the raising and lowering of camera telescoping support 40 through the depression of a button, switch, lever, and/or any combination thereof.

Lower camera support arm 42 may attach to camera telescoping support 40 at any suitable location, in embodiments, as illustrated in FIG. 1, lower camera support arm 42 may rotate three hundred and sixty degrees around telescoping support 40. This free rotation may allow an operator to position camera 46 in any suitable location. Lower camera support arm 42 may be made of any suitable material in which to support camera 46. Suitable material may be, but is not limited to, metal such as titanium, aluminum, or stainless steel, carbon fiber, fiberglass, or heavy-duty plastic. Cross-section of lower camera support arm 42 may be any suitable shape. Suitable cross-sectional shape may be, but is not limited to, circle, square, rectangle, hexagon, octagon, or i-beam. The cross-sectional length and width may be about one to ten inches. Length of the lower camera support arm may be about four inches to about thirty-six inches. Lower camera support arm 42 may connect to telescoping support 40 by any suitable mechanism. Suitable mechanism may be, but is not limited to, nuts and bolts, ball and socket fitting, press fitting, weld, adhesion, screws, rivets, clamps, latches, and/or any combination thereof. Lower camera support arm 42 may be used to provide support for camera 46. Camera 46 may be attached to lower camera support arm 42 by any suitable mechanism. Suitable mechanism may be, but is not limited to, nuts and bolts, ball and socket fitting, press fitting, weld, adhesion, screws, rivets, and/or any combination thereof. Camera 46 may pivot in any direction at the attachment area between camera 46 and lower camera support arm 42. In embodiments a curved rail 44 may be disposed on lower camera support arm 42.

Curved rail 44 may be disposed at any suitable location on lower camera support arm 42. As illustrated in FIG. 3, curved rail 44 may attach to lower camera support arm 42 by any suitable mechanism. Suitable mechanism may be, but are not limited to nuts and bolts, ball and socket fitting, press fitting, weld, adhesion, screws, rivets, clamps, latches, and/or any combination thereof. Curved rail 44 may be of any suitable shape, a suitable shape may be a crescent, circular, oval, elliptical, and/or any combination thereof. In embodiments, curved rail 44 may be any appropriate length. An appropriate length may be about one foot to about six feet. Camera 46 may be moveably disposed along curved rail 44. Camera 46 may attach to curved rail 44 by any suitable mechanism. Suitable mechanism may be, but are not limited to rollers, brackets, braces, motors, and/or any combination thereof. Motors and rollers, not illustrated, may be used to move camera 46 along curved rail 44. As illustrated in FIG. 3, during a medical procedure, if an object prevents camera 46 from viewing one or more DRAs 52, the motors may move camera 46 along curved rail 44 using rollers. This motorized movement may allow camera 46 to move to a new position that is no longer obstructed by the object without moving camera tracking system 6. While camera 46 is obstructed from viewing DRAs 52, camera tracking system 6 may send a stop signal to surgical robot 4, display 34, and/or a tablet. The stop signal may prevent SCARA 24 from moving until camera 46 has reacquired DRAs 52. This stoppage may prevent SCARA 24 and/or end effector coupler 22 from moving and/or using medical tools without being tracked by surgical system 2.

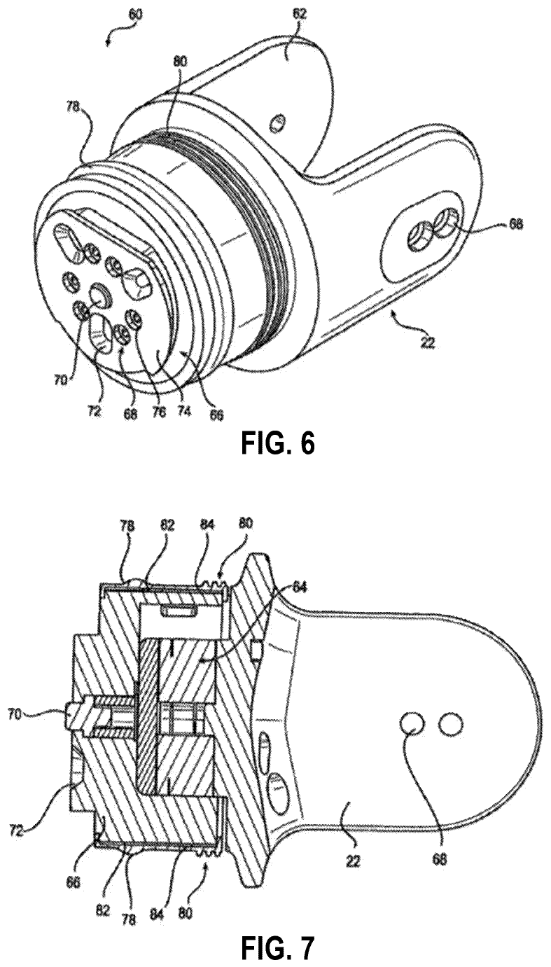

End effector coupler 22, as illustrated in FIG. 6, is configured to connect various types of passive end effectors to surgical robot 4. End effector coupler 22 can include a saddle joint 62, an activation assembly 60, a load cell 64 (FIG. 7), and a connector 66. Saddle joint 62 may attach end effector coupler 22 to SCARA 24. Saddle joint 62 may be made of any suitable material. Suitable material may be, but is not limited to metal such as titanium, aluminum, or stainless steel, carbon fiber, fiberglass, or heavy-duty plastic. Saddle joint 62 may be made of a single piece of metal which may provide end effector with additional strength and durability. The saddle joint 62 may attach to SCARA 24 by an attachment point 68. There may be a plurality of attachment points 68 disposed about saddle joint 62. Attachment points 68 may be sunk, flush, and/or disposed upon saddle joint 62. In some examples, screws, nuts and bolts, and/or any combination thereof may pass through attachment point 68 and secure saddle joint 62 to SCARA 24. The nuts and bolts may connect saddle joint 62 to a motor, not illustrated, within SCARA 24. The motor may move saddle joint 62 in any direction. The motor may further prevent saddle joint 62 from moving from accidental bumps and/or accidental touches by actively serving at the current location or passively by applying spring actuated brakes.

The end effector coupler 22 can include a load cell 64 interposed between the saddle join 62 and a connected passive end effector. Load cell 64, as illustrated in FIG. 7 may attach to saddle joint 62 by any suitable mechanism. Suitable mechanism may be, but is not limited to, screws, nuts and bolts, threading, press fitting, and/or any combination thereof

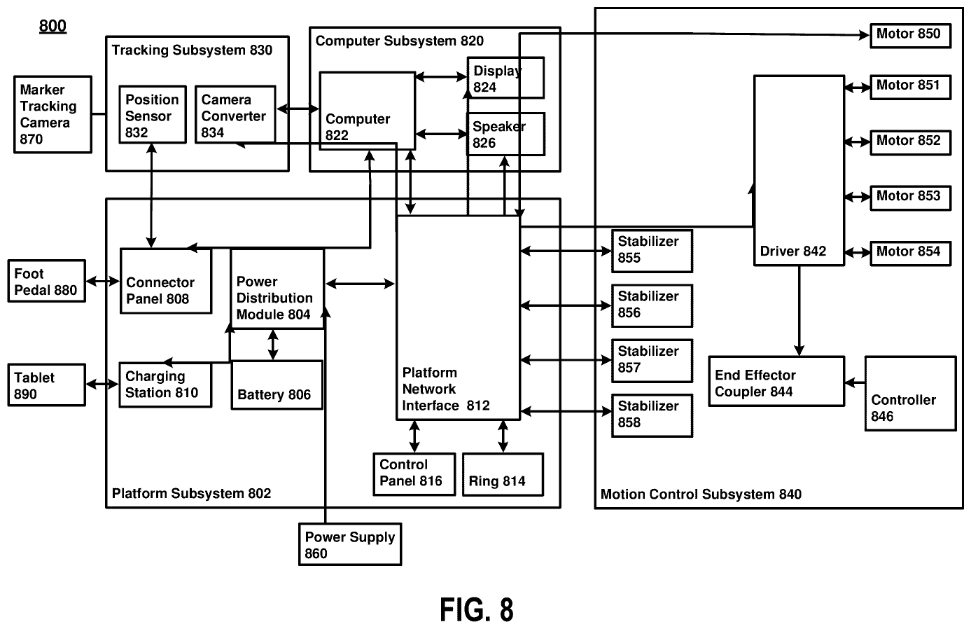

FIG. 8 illustrates a block diagram of components of a surgical system 800 according to some embodiments of the present disclosure. Referring to FIGS. 7 and 8, load cell 64 may be any suitable instrument used to detect and measure forces. In some examples, load cell 64 may be a six axis load cell, a three-axis load cell or a uniaxial load cell. Load cell 64 may be used to track the force applied to end effector coupler 22. In some embodiments the load cell 64 may communicate with a plurality of motors 850, 851, 852, 853, and/or 854. As load cell 64 senses force, information as to the amount of force applied may be distributed from a switch array and/or a plurality of switch arrays to a controller 846. Controller 846 may take the force information from load cell 64 and process it with a switch algorithm. The switch algorithm is used by the controller 846 to control a motor driver 842. The motor driver 842 controls operation of one or more of the motors. Motor driver 842 may direct a specific motor to produce, for example, an equal amount of force measured by load cell 64 through the motor. In some embodiments, the force produced may come from a plurality of motors, e.g., 850-854, as directed by controller 846. Additionally, motor driver 842 may receive input from controller 846. Controller 846 may receive information from load cell 64 as to the direction of force sensed by load cell 64. Controller 846 may process this information using a motion controller algorithm. The algorithm may be used to provide information to specific motor drivers 842. To replicate the direction of force, controller 846 may activate and/or deactivate certain motor drivers 842. Controller 846 may control one or more motors, e.g. one or more of 850-854, to induce motion of passive end effector 1100 in the direction of force sensed by load cell 64. This force-controlled motion may allow an operator to move SCARA 24 and passive end effector 1100 effortlessly and/or with very little resistance. Movement of passive end effector 1100 can be performed to position passive end effector 1100 in any suitable pose (i.e., location and angular orientation relative to defined three-dimensional (3D) orthogonal reference axes) for use by medical personnel.

Connector 66 is configured to be connectable to the base of the passive end effector 1100 and is connected to load cell 64. Connector 66 can include attachment points 68, a sensory button 70, tool guides 72, and/or tool connections/attachment points 74. Best illustrated in FIGS. 6 and 8, there may be a plurality of attachment points 68. Attachment points 68 may connect connector 66 to load cell 64. Attachment points 68 may be sunk, flush, and/or disposed upon connector 66. Attachment points 68 and 76 can be used to attach connector 66 to load cell 64 and/or to passive end effector 1100. In some examples, Attachment points 68 and 76 may include screws, nuts and bolts, press fittings, magnetic attachments, and/or any combination thereof

As illustrated in FIG. 6, a sensory button 70 may be disposed about center of connector 66. Sensory button 70 may be depressed when a passive end effector 1100 is connected to SCARA 24. Depression of sensory button 70 may alert surgical robot 4, and in turn medical personnel, that a passive end effector 1100 has been attached to SCARA 24. As illustrated in FIG. 6, guides 72 may be used to facilitate proper attachment of passive end effector 1100 to SCARA 24. Guides 72 may be sunk, flush, and/or disposed upon connector 66. In some examples there may be a plurality of guides 72 and may have any suitable patterns and may be oriented in any suitable direction. Guides 72 may be any suitable shape to facilitate attachment of passive end effector 1100 to SCARA 24. A suitable shape may be, but is not limited to, circular, oval, square, polyhedral, and/or any combination thereof. Additionally, guides 72 may be cut with a bevel, straight, and/or any combination thereof.

Connector 66 may have attachment points 74. As illustrated in FIG. 6, attachment points 74 may form a ledge and/or a plurality of ledges. Attachment points 74 may provide connector 66 a surface upon which passive end effector 1100 may clamp. In some embodiments, attachment points 74 are disposed about any surface of connector 66 and oriented in any suitable manner in relation to connector 66.

Activation assembly 60, best illustrated in FIGS. 6 and 7, may encircle connector 66. In some embodiments, activation assembly 60 may take the form of a bracelet that wraps around connector 66. In some embodiments, activation assembly 60, may be located in any suitable area within surgical system 2. In some examples, activation assembly 60 may be located on any part of SCARA 24, any part of end effector coupler 22, may be worn by medical personnel (and communicate wirelessly), and/or any combination thereof. Activation assembly 60 may be made of any suitable material. Suitable material may be, but is not limited to neoprene, plastic, rubber, gel, carbon fiber, fabric, and/or any combination thereof. Activation assembly 60 may comprise of a primary button 78 and a secondary button 80. Primary button 78 and secondary button 80 may encircle the entirety of connector 66.

Primary button 78 may be a single ridge, as illustrated in FIG. 6, which may encircle connector 66. In some examples, primary button 78 may be disposed upon activation assembly 60 along the end farthest away from saddle joint 62. Primary button 78 may be disposed upon primary activation switch 82, best illustrated on FIG. 7. Primary activation switch 82 may be disposed between connector 66 and activation assembly 60. In some examples, there may be a plurality of primary activation switches 82, which may be disposed adjacent and beneath primary button 78 along the entire length of primary button 78. Depressing primary button 78 upon primary activation switch 82 may allow an operator to move SCARA 24 and end effector coupler 22. As discussed above, once set in place, SCARA 24 and end effector coupler 22 may not move until an operator programs surgical robot 4 to move SCARA 24 and end effector coupler 22, or is moved using primary button 78 and primary activation switch 82. In some examples, it may require the depression of at least two non-adjacent primary activation switches 82 before SCARA 24 and end effector coupler 22 will respond to operator commands. Depression of at least two primary activation switches 82 may prevent the accidental movement of SCARA 24 and end effector coupler 22 during a medical procedure.

Activated by primary button 78 and primary activation switch 82, load cell 64 may measure the force magnitude and/or direction exerted upon end effector coupler 22 by an operator, i.e. medical personnel. This information may be transferred to motors within SCARA 24 that may be used to move SCARA 24 and end effector coupler 22. Information as to the magnitude and direction of force measured by load cell 64 may cause the motors to move SCARA 24 and end effector coupler 22 in the same direction as sensed by load cell 64. This force-controlled movement may allow the operator to move SCARA 24 and end effector coupler 22 easily and without large amounts of exertion due to the motors moving SCARA 24 and end effector coupler 22 at the same time the operator is moving SCARA 24 and end effector coupler 22.

Secondary button 80, as illustrated in FIG. 6, may be disposed upon the end of activation assembly 60 closest to saddle joint 62. In some examples secondary button 80 may comprise a plurality of ridges. The plurality of ridges may be disposed adjacent to each other and may encircle connector 66. Additionally, secondary button 80 may be disposed upon secondary activation switch 84. Secondary activation switch 84, as illustrated in FIG. 7, may be disposed between secondary button 80 and connector 66. In some examples, secondary button 80 may be used by an operator as a “selection” device. During a medical operation, surgical robot 4 may notify medical personnel to certain conditions by display 34 and/or light indicator 28. Medical personnel may be prompted by surgical robot 4 to select a function, mode, and/or assess the condition of surgical system 2. Depressing secondary button 80 upon secondary activation switch 84 a single time may activate certain functions, modes, and/or acknowledge information communicated to medical personnel through display 34 and/or light indicator 28. Additionally, depressing secondary button 80 upon secondary activation switch 84 multiple times in rapid succession may activate additional functions, modes, and/or select information communicated to medical personnel through display 34 and/or light indicator 28. In some examples, at least two non-adjacent secondary activation switches 84 may be depressed before secondary button 80 may function properly. This requirement may prevent unintended use of secondary button 80 from accidental bumping by medical personnel upon activation assembly 60. Primary button 78 and secondary button 80 may use software architecture 86 to communicate commands of medical personnel to surgical system 2.

FIG. 8 illustrates a block diagram of components of a surgical system 800 configured according to some embodiments of the present disclosure, and which may correspond to the surgical system 2 above. Surgical system 800 includes platform subsystem 802, computer subsystem 820, motion control subsystem 840, and tracking subsystem 830. Platform subsystem 802 includes battery 806, power distribution module 804, connector panel 808, and charging station 810. Computer subsystem 820 includes computer 822, display 824, and speaker 826. Motion control subsystem 840 includes driver circuit 842, motors 850, 851, 852, 853, 854, stabilizers 855, 856, 857, 858, end effector connector 844, and controller 846. Tracking subsystem 830 includes position sensor 832 and camera converter 834. Surgical system 800 may also include a removable foot pedal 880 and removable tablet computer 890.

Input power is supplied to surgical system 800 via a power source which may be provided to power distribution module 804. Power distribution module 804 receives input power and is configured to generate different power supply voltages that are provided to other modules, components, and subsystems of surgical system 800. Power distribution module 804 may be configured to provide different voltage supplies to connector panel 808, which may be provided to other components such as computer 822, display 824, speaker 826, driver 842 to, for example, power motors 850-854 and end effector coupler 844, and provided to camera converter 834 and other components for surgical system 800. Power distribution module 804 may also be connected to battery 806, which serves as temporary power source in the event that power distribution module 804 does not receive power from an input power. At other times, power distribution module 804 may serve to charge battery 806.

Connector panel 808 may serve to connect different devices and components to surgical system 800 and/or associated components and modules. Connector panel 808 may contain one or more ports that receive lines or connections from different components. For example, connector panel 808 may have a ground terminal port that may ground surgical system 800 to other equipment, a port to connect foot pedal 880, a port to connect to tracking subsystem 830, which may include position sensor 832, camera converter 834, and marker tracking cameras 870. Connector panel 808 may also include other ports to allow USB, Ethernet, HDMI communications to other components, such as computer 822.

Control panel 816 may provide various buttons or indicators that control operation of surgical system 800 and/or provide information from surgical system 800 for observation by an operator. For example, control panel 816 may include buttons to power on or off surgical system 800, lift or lower vertical column of support arm 16, and lift or lower stabilizers 855-858 that may be designed to engage casters (e.g., powered wheels 12) to lock surgical system 800 from physically moving. Other buttons may stop surgical system 800 in the event of an emergency, which may remove all motor power and apply mechanical brakes to stop all motion from occurring. Control panel 816 may also have indicators notifying the operator of certain system conditions such as a line power indicator or status of charge for battery 806.

Computer 822 of computer subsystem 820 includes an operating system and software to operate assigned functions of surgical system 800. Computer 822 may receive and process information from other components (for example, tracking subsystem 830, platform subsystem 802, and/or motion control subsystem 840) in order to display information to the operator. Further, computer subsystem 820 may provide output through the speaker 826 for the operator. The speaker may be part of the surgical robot, part of a head-mounted display component, or within another component of the surgical system 2. The display 824 may correspond to the display 34 shown in FIGS. 1 and 2, or may be a head-mounted display which projects images onto a see-through display screen which forms an augmented reality (AR) image that is overlaid on real-world objects viewable through the see-through display screen.

Tracking subsystem 830 may include position sensor 832 and camera converter 834. Tracking subsystem 830 may correspond to the camera tracking system 6 of FIG. 3. The marker tracking cameras 870 operate with the position sensor 832 to determine the pose of DRAs 52. This tracking may be conducted in a manner consistent with the present disclosure including the use of infrared or visible light technology that tracks the location of active or passive elements of DRAs 52, such as LEDs or reflective markers, respectively. The location, orientation, and position of structures having these types of markers, such as DRAs 52, is provided to computer 822 and which may be shown to an operator on display 824. For example, as shown in FIGS. 4 and 5, a surgical saw 1240 having a DRA 52 or which is connected to an end effector coupler 22 having a DRA 52 tracked in this manner (which may be referred to as a navigational space) may be shown to an operator in relation to a three dimensional image of a patient's anatomical structure.

Motion control subsystem 840 may be configured to physically move vertical column 16, upper arm 18, lower arm 20, or rotate end effector coupler 22. The physical movement may be conducted through the use of one or more motors 850-854. For example, motor 850 may be configured to vertically lift or lower vertical column 16. Motor 851 may be configured to laterally move upper arm 18 around a point of engagement with vertical column as shown in FIG. 2. Motor 852 may be configured to laterally move lower arm 20 around a point of engagement with upper arm 18 as shown in FIG. 2. Motors 853 and 854 may be configured to move end effector coupler 22 to provide translational movement and rotation along in about three-dimensional axes. The surgical planning computer 910 shown in FIG. 9 can provide control input to the controller 846 that guides movement of the end effector coupler 22 to position a passive end effector, which is connected thereto, with a planned pose (i.e., location and angular orientation relative to defined 3D orthogonal reference axes) relative to an anatomical structure that is to be cut during a surgical procedure. Motion control subsystem 840 may be configured to measure position of the passive end effector structure using integrated position sensors (e.g. encoders). In one of the embodiments, position sensors are directly connected to at least one joint of the passive end effector structure, but may also be positioned in another location in the structure and remotely measure the joint position by interconnection of a timing belt, a wire, or any other synchronous transmission interconnection.

FIG. 9 illustrates a block diagram of a surgical system computer platform 900 that includes a surgical planning computer 910 which may be separate from and operationally connected to a surgical robot 800 or at least partially incorporated therein according to some embodiments of the present disclosure. Alternatively, at least a portion of operations disclosed herein for the surgical planning computer 910 may be performed by components of the surgical robot 800 such as by the computer subsystem 820.

Referring to FIG. 9, the surgical planning computer 910 includes a display 912, at least one processor circuit 914 (also referred to as a processor for brevity), at least one memory circuit 916 (also referred to as a memory for brevity) containing computer readable program code 918, and at least one network interface 920 (also referred to as a network interface for brevity). The network interface 920 can be configured to connect to a C-Arm imaging device 104 in FIG. 10, an O-Arm imaging device 106 in FIG. 11, another medical imaging device, an image database 950 of medical images, components of the surgical robot 800, and/or other electronic equipment.

When the surgical planning computer 910 is at least partially integrated within the surgical robot 800, the display 912 may correspond to the display 34 of FIG. 2 and/or the tablet 890 of FIG. 8 and/or a head-mounted display, the network interface 920 may correspond to the platform network interface 812 of FIG. 8, and the processor 914 may correspond to the computer 822 of FIG. 8.

The processor 914 may include one or more data processing circuits, such as a general purpose and/or special purpose processor, e.g., microprocessor and/or digital signal processor. The processor 914 is configured to execute the computer readable program code 918 in the memory 916 to perform operations, which may include some or all of the operations described herein as being performed by a surgical planning computer.

The processor 914 can operate to display on the display device 912 an image of a bone that is received from one of the imaging devices 104 and 106 and/or from the image database 950 through the network interface 920. The processor 914 receives an operator's definition of where an anatomical structure, i.e. one or more bones, shown in one or more images is to be cut, such as by an operator touch selecting locations on the display 912 for planned surgical cuts or using a mouse-based cursor to define locations for planned surgical cuts.

The surgical planning computer 910 enables anatomy measurement, useful for knee surgery, like measurement of various angles determining center of hip, center of angles, natural landmarks (e.g. transepicondylar line, Whitesides line, posterior condylar line etc.), etc. Some measurements can be automatic while some others involve human input or assistance. This surgical planning computer 910 allows an operator to choose the correct implant for a patient, including choice of size and alignment. The surgical planning computer 910 enables automatic or semi-automatic (involving human input) segmentation (image processing) for CT images or other medical images. The surgical plan for a patient may be stored in a cloud-based server for retrieval by the surgical robot 800. During the surgery, the surgeon will choose which cut to make (e.g. posterior femur, proximal tibia etc.) using a computer screen (e.g. touchscreen) or augmented reality interaction via, e.g., a head-mounted display. The surgical robot 4 may automatically move the surgical saw blade to a planned position so that a target plane of planned cut is optimally placed within a workspace of the passive end effector interconnecting the surgical saw blade and the robot arm 20. Command enabling movement can be given by user using various modalities, e.g. foot pedal.

In some embodiments, the surgical system computer platform 900 can use two DRAs to tracking patient anatomy position: one on patient tibia and one on patient femur. The platform 900 may use standard navigated instruments for the registration and checks (e.g., a pointer similar to the one used in Globus ExcelsiusGPS system for spine surgery). Tracking markers allowing for detection of DRAs movement in reference to tracked anatomy can be used as well.

An important difficulty in knee surgery is how to plan the position of the implant in the knee and many surgeons struggle with to do it on a computer screen which is a 2D representation of 3D anatomy. The platform 900 could address this problem by using an augmented reality (AR) head-mounted display to generate an implant overlay around the actual patient knee. For example, the surgeon can be operationally displayed a virtual handle to grab and move the implant to a desired pose and adjust planned implant placement. Afterward, during surgery, the platform 900 could render the navigation through the AR head-mounted display to show surgeon what is not directly visible. Also, the progress of bone removal, e.g., depth or cut, can be displayed in real-time. Other features that may be displayed through AR can include, without limitation, gap or ligament balance along a range of joint motion, contact line on the implant along the range of joint motion, ligament tension and/or laxity through color or other graphical overlays, etc.

The surgical planning computer 910, in some embodiments, can allow planning for use of standard implants, e.g., posterior stabilized implants and cruciate retaining implants, cemented and cementless implants, revision systems for surgeries related to, for example, total or partial knee and/or hip replacement and/or trauma.

The processor 914 may graphically illustrate on the display 912 one or more cutting planes intersecting the displayed anatomical structure at the locations selected by the operator for cutting the anatomical structure. The processor 914 also determines one or more sets of angular orientations and locations where the end effector coupler 22 must be positioned so a cutting plane of the surgical saw blade will be aligned with a target plane to perform the operator defined cuts, and stores the sets of angular orientations and locations as data in a surgical plan data structure. The processor 914 uses the known range of movement of the tool attachment mechanism of the passive end effector to determine where the end effector coupler 22 attached to the robot arm 20 needs to be positioned.

The computer subsystem 820 of the surgical robot 800 receives data from the surgical plan data structure and receives information from the camera tracking system 6 indicating a present pose of an anatomical structure that is to be cut and indicating a present pose of the passive end effector and/or surgical saw tracked through DRAs. The computer subsystem 820 determines a pose of the target plane based on the surgical plan defining where the anatomical structure is to be cut and based on the pose of the anatomical structure. The computer subsystem 820 generates steering information based on comparison of the pose of the target plane and the pose of the surgical saw. The steering information indicates where the passive end effector needs to be moved so the cutting plane of the saw blade becomes aligned with the target plane and the saw blade becomes positioned a distance from the anatomical structure to be cut that is within the range of movement of the tool attachment mechanism of the passive end effector.

As explained above, a surgical robot includes a robot base, a robot arm connected to the robot base, and at least one motor operatively connected to move the robot arm relative to the robot base. The surgical robot also includes at least one controller, e.g. the computer subsystem 820 and the motion control subsystem 840, connected to the at least one motor and configured to perform operations.

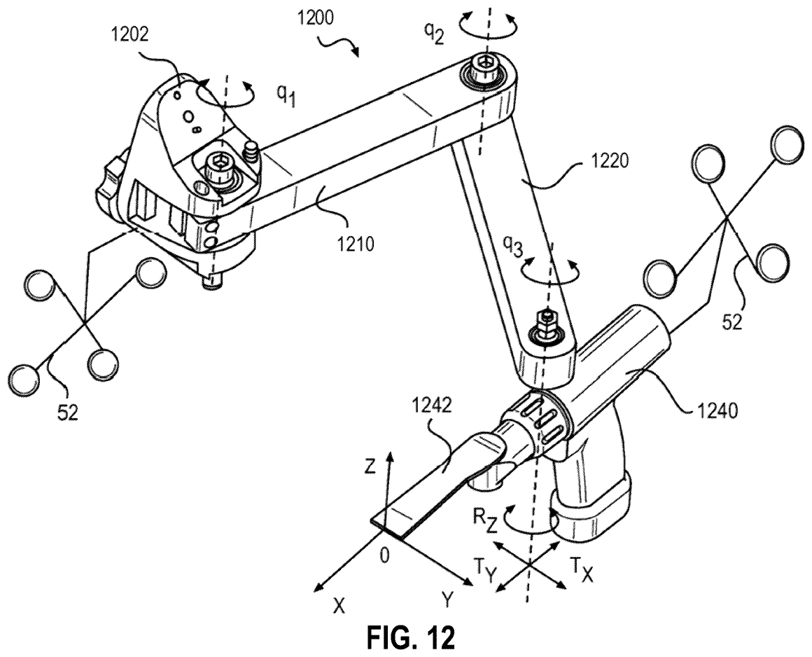

As will be explained in further detail below with regard to FIGS. 12-19, a passive end effector includes a base configured to attach to an activation assembly of the robot arm, a first mechanism, and a second mechanism. The first mechanism extends between a rotatable connection to the base and a rotatable connection to a tool attachment mechanism. The second mechanism extends between a rotatable connection to the base and a rotatable connection to the tool attachment mechanism. The first and second mechanisms pivot about the rotatable connections which may be configured to constrain movement of the tool attachment mechanism to a range of movement within a working plane. The rotatable connections may be pivot joints allowing 1 degree-of-freedom (DOF) motion, universal joints allowing 2 DOF motions, or ball joints allowing 3 DOF motions. The tool attachment mechanism is configured to connect to the surgical saw comprising a saw blade for cutting. The first and second mechanisms may be configured to constrain a cutting plane of the saw blade to be parallel to the working plane.

In some embodiments, the operations performed by the at least one controller of the surgical robot also includes controlling movement of the at least one motor based on the steering information to reposition the passive end effector so the cutting plane of the saw blade becomes aligned with the target plane and the saw blade becomes positioned the distance from the anatomical structure to be cut that is within the range of movement of the tool attachment mechanism of the passive end effector. The steering information may be displayed to guide an operator's movement of the surgical saw and/or may be used by the at least one controller to automatically move the surgical saw.

In one embodiment, the operations performed by the at least one controller of the surgical robot also includes providing the steering information to a display device for display to guide operator movement of the passive end effector so the cutting plane of the saw blade becomes aligned with the target plane and so the saw blade becomes positioned the distance from the anatomical structure, which is to be cut, that is within the range of movement of the tool attachment mechanism of the passive end effector. The display device may correspond to the display 824 (FIG. 8), the display 34 of FIG. 1, and/or a head-mounted display.

For example, the steering information may be displayed on a head-mounted display which projects images onto a see-through display screen which forms an augmented reality image that is overlaid on real-world objects viewable through the see-through display screen. The operations may display a graphical representation of the target plane with a pose overlaid on a bone and with a relative orientation there between corresponding to the surgical plan for how the bone is planned to be cut. The operations may alternatively or additionally display a graphical representation of the cutting plane of the saw blade so that an operator may more easily align the cutting plane with the planned target plane for cutting the bone. The operator may thereby visually observe and perform movements to align the cutting plane of the saw blade with the target plane so the saw blade becomes positioned at the planned pose relative to the bone and within a range of movement of the tool attachment mechanism of the passive end effector.

An automated imaging system can be used in conjunction with the surgical planning computer 910 and/or the surgical system 2 to acquire pre-operative, intra-operative, post-operative, and/or real-time image data of a patient. Example automated imaging systems are illustrated in FIGS. 10 and 11. In some embodiments, the automated imaging system is a C-arm 104 (FIG. 10) imaging device or an O-arm® 106 (FIG. 11). (O-arm® is copyrighted by Medtronic Navigation, Inc. having a place of business in Louisville, Colo., USA) It may be desirable to take x-rays of a patient from a number of different positions, without the need for frequent manual repositioning of the patient which may be required in an x-ray system. C-arm 104 x-ray diagnostic equipment may solve the problems of frequent manual repositioning and may be well known in the medical art of surgical and other interventional procedures. As illustrated in FIG. 10, a C-arm includes an elongated C-shaped member terminating in opposing distal ends 112 of the “C” shape. C-shaped member is attached to an x-ray source 114 and an image receptor 116. The space within C-arm 104 of the arm provides room for the physician to attend to the patient substantially free of interference from the x-ray support structure.

The C-arm is mounted to enable rotational movement of the arm in two degrees of freedom, (i.e. about two perpendicular axes in a spherical motion). C-arm is slidably mounted to an x-ray support structure, which allows orbiting rotational movement of the C-arm about its center of curvature, which may permit selective orientation of x-ray source 114 and image receptor 116 vertically and/or horizontally. The C-arm may also be laterally rotatable, (i.e. in a perpendicular direction relative to the orbiting direction to enable selectively adjustable positioning of x-ray source 114 and image receptor 116 relative to both the width and length of the patient). Spherically rotational aspects of the C-arm apparatus allow physicians to take x-rays of the patient at an optimal angle as determined with respect to the particular anatomical condition being imaged.

The O-arm® 106 illustrated in FIG. 11 includes a gantry housing 124 which may enclose an image capturing portion, not illustrated. The image capturing portion includes an x-ray source and/or emission portion and an x-ray receiving and/or image receiving portion, which may be disposed about one hundred and eighty degrees from each other and mounted on a rotor (not illustrated) relative to a track of the image capturing portion. The image capturing portion may be operable to rotate three hundred and sixty degrees during image acquisition. The image capturing portion may rotate around a central point and/or axis, allowing image data of the patient to be acquired from multiple directions or in multiple planes.

The O-arm® 106 with the gantry housing 124 has a central opening for positioning around an object to be imaged, a source of radiation that is rotatable around the interior of gantry housing 124, which may be adapted to project radiation from a plurality of different projection angles. A detector system is adapted to detect the radiation at each projection angle to acquire object images from multiple projection planes in a quasi-simultaneous manner. The gantry may be attached to a support structure O-arm® support structure, such as a wheeled mobile cart with wheels, in a cantilevered fashion. A positioning unit translates and/or tilts the gantry to a planned position and orientation, preferably under control of a computerized motion control system. The gantry may include a source and detector disposed opposite one another on the gantry. The source and detector may be secured to a motorized rotor, which may rotate the source and detector around the interior of the gantry in coordination with one another. The source may be pulsed at multiple positions and orientations over a partial and/or full three hundred and sixty degree rotation for multi-planar imaging of a targeted object located inside the gantry. The gantry may further comprise a rail and bearing system for guiding the rotor as it rotates, which may carry the source and detector. Both and/or either O-arm® 106 and C-arm 104 may be used as automated imaging system to scan a patient and send information to the surgical system 2.

Images captured by the automated imaging system can be displayed a display device of the surgical planning computer 910, the surgical robot 800, and/or another component of the surgical system 2.

Although embodiments herein may be described in regards to an EFlex-TKA system for knee surgery similar embodiments are applicable to other surgical robot systems used for other robotic assisted surgeries.

In some embodiments, the surgical robot system is EFlex-TKA system that controls, includes, or supports an EFlex station and the procedure guides a user to optimally position and orient the EFlex station around the operating table with a tracked patient positioned on it to ensure that a surgeon can perform all the required resections to execute a planned Total Knee Arthroplasty (“TKA”) surgery without the need of repositioning the EFlex station in between the required resections (e.g., because all of the resections are within reach of a robotic arm of the EFlex station).

An EFlex-TKA system is a robotic system for knee surgery. It can be used to prepare the bones (tibia and femur) on which implants will be placed. The robotic system integrates a 6 active Degrees of Freedom (“DOF”) serial arm on which a passive End-Effector Arm (“EEA”) structure guiding the saw blade is mounted. At the end of this passive structure a sagittal saw is attached. Before each resection, the robotic arm brings the sawblade towards the planned resection plane in patient anatomy and positions it within this planned resection plane before execution of resection. To execute the resection, the surgeon holds the sagittal saw and cuts the bones while eventually watching on the navigation system (or Augmented Reality (“AR”) headset) displaying any type of relevant feedback or information associated with the procedure.

There are multiple patient anatomy registration workflows available for use with the system. Some workflows require pre-operative scans or images of the patient (e.g., X-ray,

Computerized Tomography (“CT”)). On the other hand, the imageless workflow does not require any pre-operative images. To obtain intra-operative information about the patient anatomy, the surgeon measures key parameters of the bone using an intra-operative tracking system and appropriate tracked instrument to capture points on patient anatomy. Later, this information is used to plan the implant position and orientation with respect to patient anatomy and navigate robot and surgical instruments during bone resection and implant placement.

After patient registration and implants planning on the EFlex-TKA Software, the EFlex Station including the serial robotic arm with the EEA attached on the palm and EEA reference element connected to the EEA is brought by the OR staff at the selected side of the operating room table, either on lateral side or on the contra-lateral side (opposite side of operated leg)) based on the operating room set up and is stabilized on the floor.

Once stabilized, the serial robotic arm is brought automatically to the suitable position for the surgery on the surgeon's request (e.g., presses a foot pedal, touchscreen/AR interaction). The sawblade guided by the End Effector Arm passive structure and actuated by the dedicated sagittal saw handpiece then allows the surgeon to precisely remove bone in the cutting plane.

Embodiments associated with selecting an optimal position of a surgical robot system for a surgery are described below.

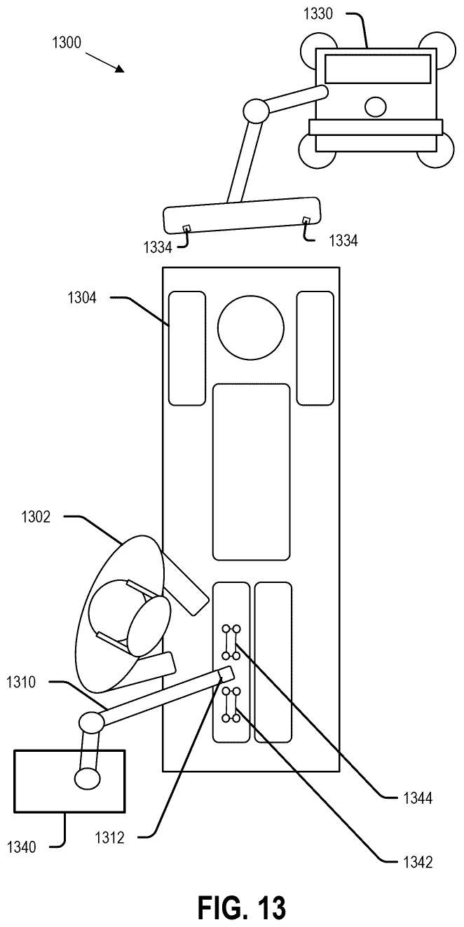

FIG. 13 is an overhead view of a surgical robot system 1300 arranged during a surgical procedure in a surgical room. In this example the surgical robot system 1300 includes a camera tracking system 1330 for determining a pose (e.g., position and/or orientation) of one or more objects (e.g., the surgeon 1302, patient 1304, and/or medical equipment) in the surgical room. The surgical robot system 1300 further includes a surgical robot station 1340, which can provide robotic assistance according to some embodiments. The surgical robot station 1340 includes a robot arm 1310 holding a surgical tool 1312. In some examples, the robot arm can include an end-effector structure and/or an end effector reference element which can include one or more tracking fiducials. A patient reference element 1342, 1344 (“DRB”) can have a plurality of tracking fiducials and be secured directly to the patient 1304 (e.g., to a bone of the patient). In this example, a femur reference marker 1344 and a tibia reference marker 1342 are attached to the patient and allow the camera tracking system 1330 to determine a location of the patient 1302 (and more specifically a surgical site on the patient 1302).

The camera tracking system 1330 includes tracking cameras 1334 which may be spaced apart stereo cameras configured with partially overlapping field-of-views. The camera tracking system 1330 can have any suitable configuration of arm(s) to move, orient, and support the tracking cameras 1334 in a desired location, and may contain at least one processor operable to track location of an individual fiducial and pose of an array of fiducials of a reference element.

As used herein, the term “pose” refers to the location (e.g., along 3 orthogonal axes) and/or the rotation angle (e.g., about the 3 orthogonal axes) of an object (e.g., a fiducials or DRB) relative to another object (e.g., another fiducial or surveillance fiducial) and/or to a defined coordinate system (e.g., a camera coordinate system, a navigation coordinate system, etc.). A pose may therefore be defined based on only the multidimensional location of the fiducials relative to another fiducial and/or relative to the defined coordinate system, based on only the multidimensional rotational angles of the fiducials relative to the other fiducial and/or to the defined coordinate system, or based on a combination of the multidimensional location and the multidimensional rotational angles. The term “pose” therefore is used to refer to location, rotational angle, or combination thereof.

The tracking cameras 1334 may include, e.g., infrared cameras (e.g., bifocal or stereophotogrammetric cameras), operable to identify, for example, active and passive tracking fiducials for single fiducials (e.g., surveillance fiducial) and reference elements which can be formed on or attached to the patient (e.g., patient reference element, DRB, etc.), end effector (e.g., end effector reference element), XR headset(s) worn by a surgeon 1302 and/or a surgical assistant, etc. in a given measurement volume of a camera coordinate system while viewable from the perspective of the tracking cameras 1334. The tracking cameras 1334 may scan the given measurement volume and detect light that is emitted or reflected from the fiducials in order to identify and determine locations of individual fiducials and poses of the reference elements in three-dimensions. For example, active reference elements may include infrared-emitting fiducials that are activated by an electrical signal (e.g., infrared light emitting diodes (“LEDs”)), and passive reference elements may include retro-reflective fiducials that reflect infrared light (e.g., they reflect incoming IR radiation into the direction of the incoming light), for example, emitted by illuminators on the tracking cameras 1334 or other suitable device.

In additional or alternative embodiments, a surgical robot system may be separate from a camera tracking system and/or a surgical robot station and communicate with them via wired or wireless communication.

In the example of FIG. 13, the surgical robot station 1340 is an Eflex station positioned to assist with a TKA. The surgical robot station 1340 includes a robotic arm 1310 which may include an End-Effector Arm (“EEA”) structure that can hold the surgical tool 1312 (e.g., a sagittal saw). The EEA may also include a reference marker that can be detected by the camera tracking system 1330 to determine a location of the robotic arm 1310 and/or the surgical robot station 1340. Accordingly, the camera tracking system is able to provide a pose (also referred to herein as a position) of the surgical robot station 1310 relative to the patient 1304.

Some embodiments herein refer to movement of the robotic arm 1310. The movement of the robotic arm 1310 can refer to movement of a portion of the robotic arm connected to the EEA and/or movement of the EEA itself. In some examples, the movement of the robotic arm 1310 can include any change in pose (e.g., 3D movement as well as 3D rotation) of the robotic arm 1310, EEA, and/or the surgical tool 1312.

In some embodiments, after patient registration, when the EFlex station is brought at the selected side of the operating room table, the EFlex-TKA system can determine the optimal position and orientation of the EFlex Station with respect to patient anatomy. The EFlex-TKA system can receive a continuous indication of a real time location in space by the navigation system (e.g., ExcelsiusHub) of: patient anatomy (e.g., obtained via tracking of tibia and femur reference elements); the EFlex station (e.g., obtained via tracking of the EEA reference element attached to the robotic arm). The EFlex-TKA system can further receive a continuous indication of an operating room set up defining a positioning of the EFlex station with respect to the operating room table (e.g., either on lateral side (side of operated leg) or on the contra-lateral side (opposite side of operated leg)).

With these inputs and considering the geometric parameters of both the EFlex serial robotic arm and serial kinematics of the end effector arm, the EFlex-TKA software calculates a set of EFlex station locations/orientations from where all resection planes are reachable by the EFlex station. These locations are calculated by computing the robotic arm joints parameters needed to achieve desired position and orientation of the end effector arm required for all planned resections and ensuring that it is within EFlex station's workspace in current patient anatomy location using inverse kinematics model. This calculation results in the determination of an area in space including all the EFlex station locations/orientations being a solution of the inverse kinematics calculation in a form of a heatmap.

The optimal location/orientation across all solutions (e.g., represented in a green area of a heatmap) can then be determined by calculating and extracting the barycenter of the surface (e.g., the center of gravity of the surface). This determined optimal location/orientation of the EFlex Station with respect to patient anatomy is then communicated to user via the graphical user interface of EFlex-TKA software application, with active guidance provided to bring the EFlex Station towards the optimal location around the operating table.

Embodiments associated with guiding a surgical robot system to an optimal position for a surgery are described below.

During placement of the EFlex station on casters around the operating table, the EFlex-TKA software guides the user via the graphical user interface to bring the EFlex station towards the optimal position and orientation of the EFlex station around the operating table by displaying the target optimal location/orientation with respect to operating table on the navigation system monitor (or Augmented Reality (“AR”) headset if available) and the direction from actual EFlex station location/orientation to this optimal target location/orientation. During this positioning procedure, the graphical user interface can also provide the user the required limb flexion angle for appropriate determination of optimal target location, and the real time distance from the current EFlex station location to optimal location.

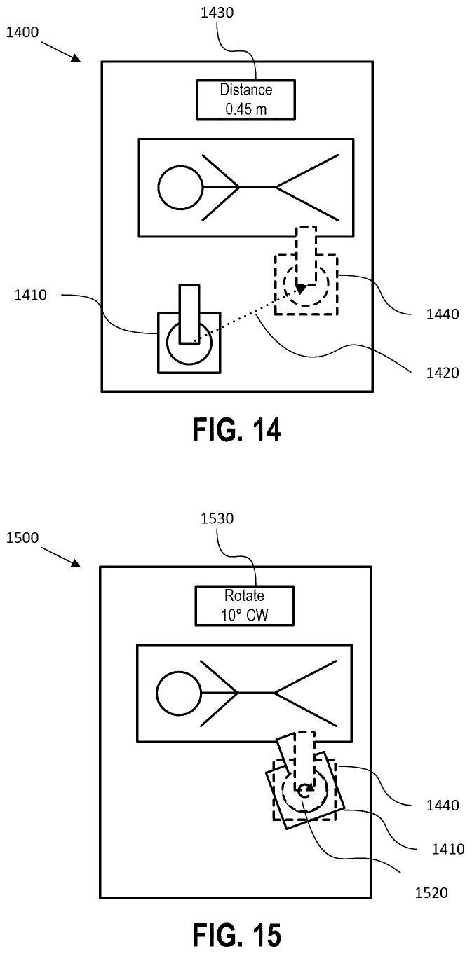

FIG. 14 illustrates an example of a display 1400 that may be output by the surgical robot system. The display 1400 includes an indication of a current position 1410 of a surgical robot station (e.g., the current position may be relative to a patient on an operating table, an anatomy of the patient, and/or another reference point in the surgery environment). The display 1400 further includes an indication of a desired position 1440 of the surgical robot station as well. The display 1400 further includes an indication of a direction 1420 and a distance 1430 to move the surgical robot station 1440 from its current position 1410 to the desired position 1440.

Even though the EFlex station might be detected on the determined optimal target location with a defined tolerance, it may not have the optimal orientation. In such case, the EFlex-TKA software guides the user via the graphical user interface to bring the EFlex station towards the optimal orientation of the EFlex station with respect to the operating table by displaying the target optimal orientation with respect to operating table together with appropriate visual on the navigation system monitor (or Augmented Reality (“AR”) headset if available).

FIG. 15 illustrates an example of a display 1500 that may be output by the surgical robot system. As in display 1400, the display 1500 includes an indication of a current position 1410 of a surgical robot station. However, rather than a direction and/or distance to move the surgical robot station, the display 1500 further includes an indication of a direction 1520 and an angle 1530 to rotate the surgical robot station to achieve a desired orientation of the desired position.

Once the EFlex station is detected on the determined optimal target location/orientation with a defined tolerance, the user is notified by the EFlex-TKA software via appropriate visuals on the graphical user interface. The EFlex Station outline in the graphical user interface turns green when the system is positioned correctly around operating table.

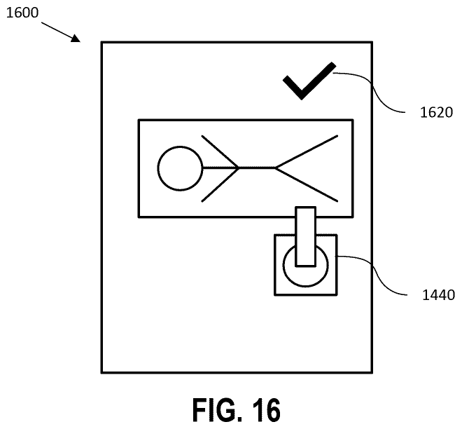

FIG. 16 illustrates an example of a display 1600 that may be output by the surgical robot system. Display 1600 includes an indication 1620 that the current position (e.g., both location and orientation) of the surgical robot station is at the desired position 1440.

In such conditions, the user is now allowed to deploy stabilizers of EFlex station to stabilize the robotic station on the floor and progress with surgical procedure, being ensured that EFlex station is properly positioned and oriented with respect to patient so that all planned resection planes are reachable by the EFlex-TKA sawblade and within the workspace of the EFlex station robotic arm. Once the EFlex station is properly stabilized on the floor, the user is notified by the EFlex-TKA software via appropriate visuals on the graphical user interface. A green checkmark can be displayed in the center of the EFlex station outline.

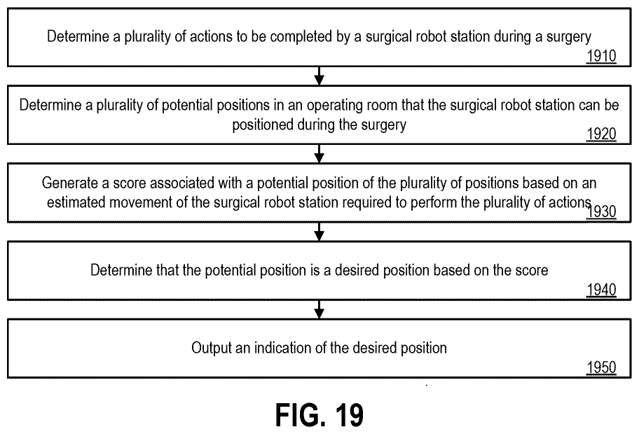

FIG. 19 is a flow chart illustrating an example of operations that can be performed by a surgical robot system to determine a desired position of a surgical robot station and guide the surgical robot station to the desired position. Herein the operations will be described as being caused by the processor 914 of the surgical system computer platform 900 executing program code 918 stored in in memory 916. However, the operations can be performed by any suitable processing circuitry of a surgical robot system.

During any of the steps in FIG. 19, the processor 914 receives a continuous indication of where the patient is based on the DRBs 1342,1344 and where the surgical robot station 1340 is based on the DRA 52 which are tracked by the cameras 46, and constantly displays an updated, real-time robot station location on the display 1400 as shown in FIGS. 14-16.

In essence, the processor 914 determines the position of the patient and/or therefore the operating table, determines an optimal position of the robot station to perform all of the robot assisted surgical functions, and displays the determined position as well as the current position of the robot station. The graphical display showing the actual and optical location of the robot station is updated in real-time as the user moves the station to its optimal position.

At block 1910, processor 914 determines a plurality of actions to be completed by a surgical robot station during a surgery.

At block 1920, processor 914 determines a plurality of potential positions in an operating room that the surgical robot station can be positioned during the surgery. In some embodiments, each potential position of the plurality of positions includes a potential location and a potential orientation of the surgical robot station in the operating room.

At block 1930, processor 914 generates a score associated with a potential position of the plurality of positions based on an estimated movement of the surgical robot station required to perform the plurality of actions. In some embodiments, the surgical robot station includes a base configured to stabilize the surgical robot station at a desired position in the operating room and a robotic arm configured to hold a surgical tool at a plurality of different poses within a range of the robotic arm, each pose of the plurality of different poses having a different pose. The plurality of actions to be completed by the surgical robot station includes positioning the surgical tool at a plurality of poses relative to a patient. The operation to generate the score includes to determine the score based on at least one of: 1) an estimated ability of the robotic arm to position the surgical tool at each pose of the plurality of poses relative to the patient; 2) an estimated amount of movement of the robotic arm to position the surgical tool at each pose of the plurality of poses relative to the patient; 3) an estimated type of movement of the robotic arm to position the surgical tool at each pose of the plurality of poses relative to the patient; 4) an estimated stability of the robotic arm while holding the surgical tool at each pose of the plurality of poses relative to the patient; 5) a degree to which the EFlex serial robotic arm and/or the end effector arm approach a singular configuration (a singular configuration is a specific arm position where the robot arm can't move freely when the robot's joints align in a way that prevents smooth motion) and 6) an estimated angle of a joint associated with the robotic arm used to position the surgical tool at each pose of the plurality of poses relative to the patient.

In additional or alternative embodiments, the operation to generate the score includes to quantify characteristics of simulated movement of the robotic arm performing the plurality of actions while the surgical robot station is positioned at the potential position.

In additional or alternative embodiments, the operation to generate the score includes to determine a plurality of scores that are each associated with one potential position of the plurality of potential positions.

At block 1940, processor 914 determines that the potential position is a desired position based on the score. In some embodiments, the operation to determine that the potential position is the desired position of the surgical robot station includes determining that the potential position is a center of gravity of a surface defined by the plurality of scores.

At block 1950, processor 914 outputs an indication of the desired position. In some embodiments, the operation to output the indication of the desired position includes to: generate a heat map from the surface defined by the plurality of scores; and display the heat map with a graphical element indicating the desired position.

In additional or alternative embodiments, the operation to output the indication of the desired position includes to: determine a current position of the surgical robot station; and output an indication of the current position of the surgical tool relative to the desired position of the surgical tool. In some examples, the operation to determine the current position of the surgical robot station includes to receive an indication of the current position from a camera tracking system.

In additional or alternative examples, the operation to output the indication of the current position of the surgical robot station includes at least one of: 1) display a virtual map including a virtual element representing the current position of the surgical tool and a virtual element representing the desired position of the surgical tool; 2) output an indication that the surgical tool is positioned at the desired position; 3) output an indication of a direction from the current position to the desired position; 4) output an indication of a distance between the current position and the desired position; and 5) transmit instructions to cause the surgical tool to move to the desired position.

Various operations from the flow chart of FIG. 19 may be optional with respect to some embodiments of surgical robot systems and related methods.

Although not illustrated in FIG. 19, in some embodiments, the score is a first score and the potential position is a first potential position. The surgical robot system can further determine a second score associated with an ability of the surgical tool to perform a first portion of the plurality of actions at a second potential position of the plurality of potential positions and an ability of the surgical tool to perform a second portion of the plurality of actions at a third potential position of the plurality of potential positions. Furthermore, the surgical robot system can determine whether to split the operation into a plurality of segments based on the first score and/or the second score, each segment of the plurality of segments being associated with a different portion of the plurality of actions to be completed by the surgical tool.

In some examples, the operation to determine whether to split the operation into the plurality of segments includes to determine to split the operation into the plurality of segments. The surgical robot system can further, prior to the first segment, determine a first current position of the surgical tool. The surgical robot system can further, prior to the first segment, output an indication of the first current position of the surgical tool relative to the second potential position of the surgical tool. The surgical robot system can further, subsequent to the first segment and prior to the second segment, determine a second current position of the surgical tool. The surgical robot system can further, subsequent to the first segment and prior to the second segment, output an indication of the second current position of the surgical tool relative to the third potential position of the surgical tool.

Embodiments associated with dynamically updating a path of a robotic arm of a surgical robot system are described below.

Once stabilized, the sawblade attached to the distal end of the End Effector Arm is automatically positioned by the robotic arm at the selected resection plane upon the surgeon's command (e.g., pressing a foot pedal, interacting via touchscreen, or using AR). This movement uses the EFlex Station Move Mode (referred to as “go-to-plane” mode).

Guided by the passive structure of the End Effector Arm and actuated by the dedicated sagittal saw handpiece, the sawblade enables the surgeon to precisely remove bone along the cutting plane. The sawblade is actively maintained on the planned resection plane relative to patient anatomy through active tracking, using the “real-time compensation” mode.

Upon completing the resection and locking the End Effector Arm, the surgeon can select the next resection to be performed. The sawblade, attached to the distal end of the End Effector Arm, is then automatically repositioned by the EFlex station robotic arm to the newly selected resection plane.

The robotic arm's dynamic motion to resection planes (tibia proximal, femur distal, femur posterior, femur posterior chamfer, femur anterior, femur anterior chamfer) involves the automatic movement of the robotic arm to accurately position the sawblade at the selected resection. Following the successful positioning of the EFlex Station around operating table, the user can select the next resection to be performed via the graphical user interface of EFlex-TKA Software.

Once the next resection to be executed is selected, a notification is provided to the user on the graphical user interface of EFlex-TKA Software to request the EFlex foot pedal to be pressed to enable the robotic arm motion and the LED indicator on the End Effector Arm turns blue. The movement of the EFlex Station robotic arm is enabled and controlled by the user via the EFlex Foot Pedal or alternative move enable switch on EFlex Main Panel until the selected planned resection plane has been reached by the sawblade. As soon as the sawblade reaches the target resection plane, a notification about dynamic motion to selected resection plane being successfully completed is displayed to the user on the graphical user interface of EFlex-TKA Software, the LED indicator on the End Effector Arm turns green and Active Tracking Mode (“real time compensation” mode) is initiated if or until all safety conditions to enable this mode are met. Active Tracking Mode is used to actively update position and orientation of the attached sawblade with respect to patient anatomy to track a changing target resection plane, accounting for patient bone movement during resection.

In any robotic arm motion, the principle of continuous activation is applied, which means that movement only occurs when the user continuously presses the robotic arm move enable switch. If during the robotic arm motion user releases the EFlex foot pedal, motion is immediately stopped. In summary, Dynamic Motion to Resection Planes (Move Mode) can be stopped by: releasing the foot pedal or foot pedal alternate button at any time; covering the relevant patient DRB (either the Tibia or Femur Arrays, based on the selected target resection), such that the arrays cannot be properly tracked by the camera; or pressing the Emergency Stop button on the EFlex Station.

Move Mode is used to move the End Effector Arm from a fixed known location to another fixed predefined location. In this mode, the robotic arm motion is performed along a defined path. As described above, Move Mode is typically used during Dynamic Motion to Resection Planes to bring the sawblade towards the planned resection plane before execution of resection, or move the robot away from the patient between resections. As Move Mode is an automatically generated motion, it is critical for the robotic arm to know about its environment to avoid collisions.

As the End Effector Arm assembled on EFlex station robotic arm is close to the patient and various OR equipment, and as movements between the different resections planes can be significant depending on the selected resection sequence, a Collision Avoidance algorithm has been implemented. The Collision Avoidance allows to define and calculate a path for the robotic arm considering the End Effector Arm and items attached to it (e.g. saw handpiece, sawblade, battery, EEA Reference Element) in the way that it avoids collisions with other elements of the environment (e.g. patient anatomy as well as Tibia and Femur Arrays, Operating Room table, robot station, anesthesia drape). All these elements are modelized in a simplified way in the surgery scene, as shown in FIGS. 5 and 13. The surgery scene is being updated in real time in the EFlex-TKA software backend, as relative position of the relevant elements is being updated via navigation information of the relevant arrays (52, 1342, 1344) by the navigation station (e.g., the hip center) and some objects within the scene (anesthesia drape, OR Table plane) are modelized in assumed localization compared to registered and tracked anatomical landmarks. Scene definition can be simplified for easier and faster calculations but shall at the same time have sufficient resolution to be representative for appropriate robotic arm path calculation.

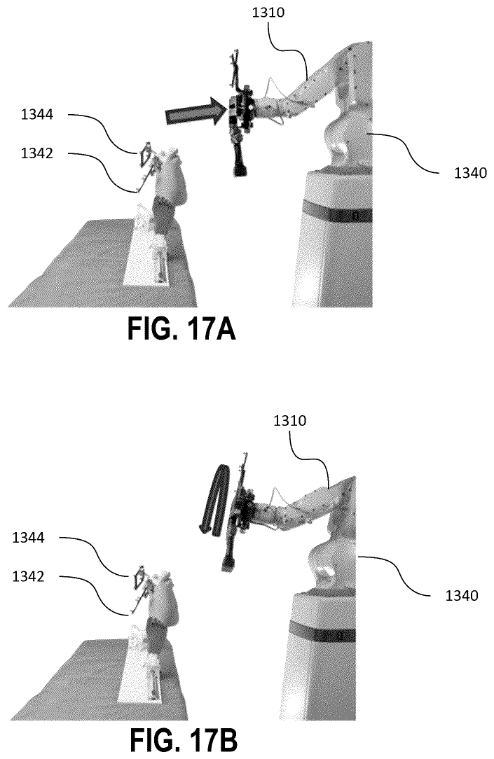

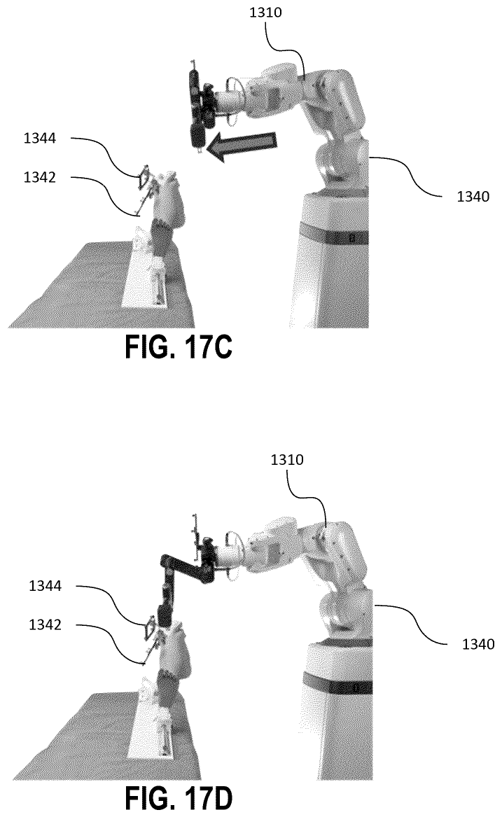

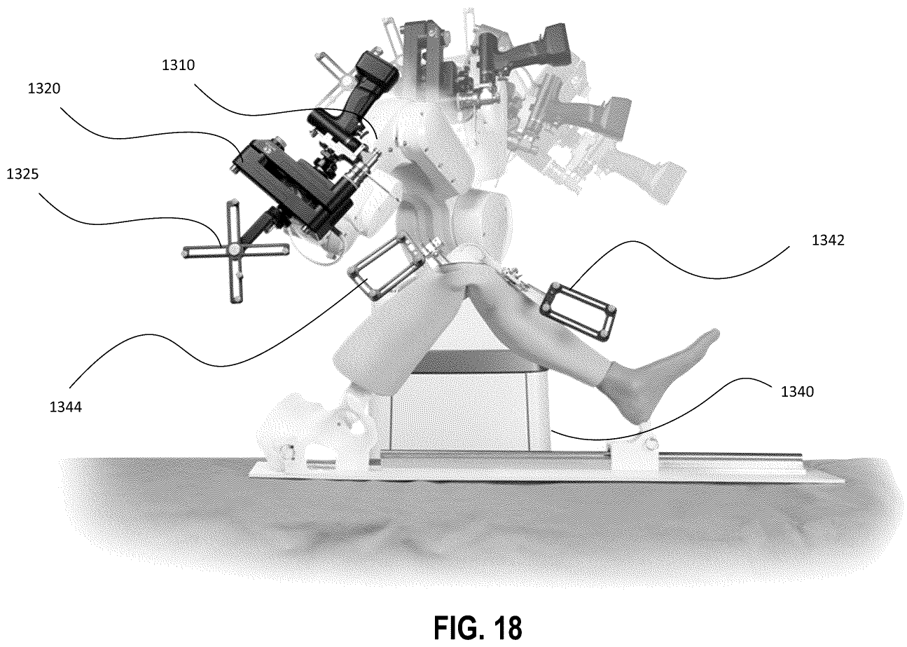

During path planning of Dynamic Motion to Resection Planes, the potential collisions with the environment are determined by the Collision Avoidance algorithm. The path between initial and target robotic arm positions is then defined and calculated so that there are no collisions between End Effector Arm and items attached to it and the environment. In addition to the Collision Avoidance for path planning algorithm, other rules are defined to constrain robotic arm path planning and definition. Therefore, when the End Effector Arm assembled on EFlex station robotic arm is transitioning between resection planes, built-in safety measures ensure that rotations happen away from patient anatomy while constantly evaluating the possibility of collisions using Collision Avoidance. An example sequence of robotic arm motion during Dynamic Motion to Resection Planes is provided in FIGS. 17A-D as well as FIG. 18. These figures are described below in terms of robotic arm 1310, however, the term is used to refer to a robotic arm and an end effector arm (the innovations can apply to just the robotic arm, just the end effector arm, or the combination of robotically controlled moving parts of a surgery robot station).

In FIG. 17A, the robotic arm 1310 of the surgical robot station 1340 is moved away from the patient anatomy (e.g. identified by femur reference marker 1344 and tibia reference marker 1342). In FIG. 17B, the robotic arm 1310 rotates to align the sawblade to the next selected resection plane. In FIG. 17C, while completing rotation of the robotic arm 1310 to align the sawblade to the next selected resection plane, the robotic arm 1310 is translated back closer to patient anatomy. In FIG. 17D, the sawblade is aligned to the target resection plane and active tracking is initiated. At this point, a surgeon can perform the resection while the robotic arm 1310.

As an example, FIG. 18 shows transition between the tibial resection and the femoral distal resection, which is the transition exhibiting the largest rotation of the robotic arm 1310 (e.g., the end effector arm).

Note that in case of potential collision detected by the Collision Avoidance algorithm during Dynamic Motion to Resection Planes, the dynamic motion to selected resection plane is stopped and a notification about collision being detected is displayed to the user on the graphical user interface of EFlex-TKA Software and the LED indicator on the End Effector Arm turns red.

Some of these embodiments can provide one or more advantages. In some examples, procedures enable safe robotic arm motion to transition the End Effector Arm and its attached sawblade from the current resection plane to the next selected plane prior to executing the resection, including a procedure for moving the robotic arm away from the patient between resections to execute required robotic arm configuration changes. In additional or alternative examples, real-time update of the surgery scene, modeling a virtual environment of the EFlex robotic arm that can be used to detect collision between End Effector Arm and attached accessories and the environment. In additional or alternative examples, a collision Avoidance algorithm and its integration within the EFlex robotic arm path planning calculations to ensure safe and accurate operation.

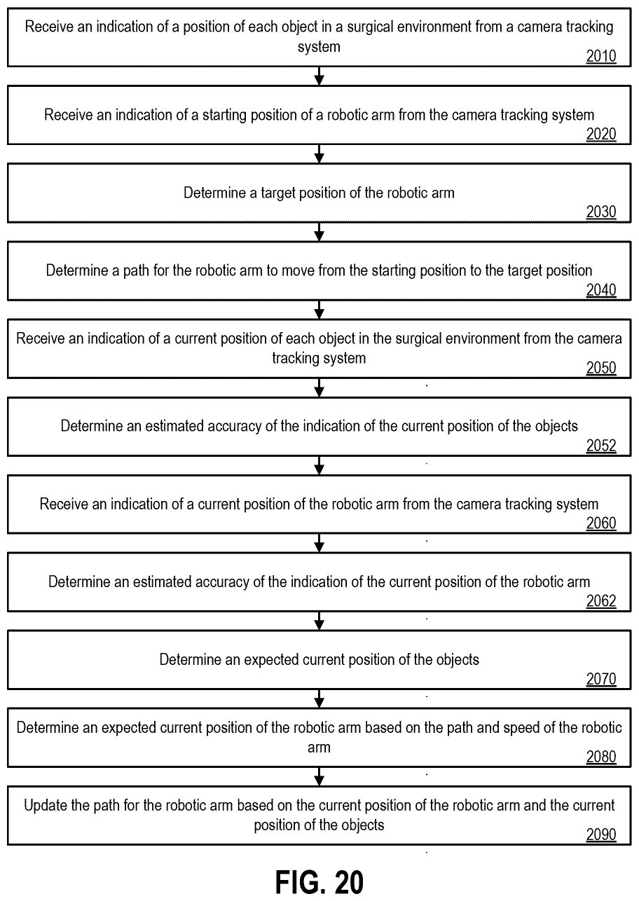

FIG. 20 is a flow chart illustrating an example of operations that can be performed by a surgical robot system to dynamically update a path of a robotic arm 1310 of a surgical robot station 1340. Herein the operations will be described as being caused by the processor 914 of the surgical system computer platform 900 executing program code 918 stored in in memory 916. However, the operations can be performed by any suitable processing circuitry of a surgical robot system.

As in FIG. 19, during any of the steps in FIG. 20, the processor 914 receives a continuous indication of where the patient is based on the DRBs 1342, 1344 and where the surgical robot station 1340 is based on the DRA 52 which are tracked by the cameras 46, and all steps of FIG. 20 reference those positions to perform the steps.

At block 2010, processor 914 receives an indication of a position of each object in a surgical environment from a camera tracking system.

At block 2020, processor 914 receives an indication of a starting position of a robotic arm from the camera tracking system.

At block 2030, processor 914 determines a target position of the robotic arm.

At block 2040, processor 914 determines a path for the robotic arm to move from the starting position to the target position. In some embodiments, the operation to determine the path for the robotic arm includes to: determine a proximity threshold associated with each object of the first plurality of objects; and determine the path for the robotic arm so that the path avoids passing within the proximity threshold of each object of the first plurality of objects.

At block 2050, processor 914 receives an indication of a current position of each object in the surgical environment from the camera tracking system. In some embodiments, these objects include the same and/or different objects than were detected in block 2010. In some examples, the first plurality of objects includes objects within a threshold a threshold distance of the robotic arm at a first time, and the second plurality of objects includes objects within a threshold distance of the robotic arm at a second time.

In additional or alternative embodiments, the operation to receive the indication of the current position of the second plurality of objects comprises at least one of: periodically receive the indication of the current position of the second plurality of objects in the surgical environment from the camera tracking system; receive the indication of the current position of the second plurality of objects in the surgical environment from the camera tracking system in response to detecting a collision; and receive the indication of the current position of the second plurality of objects in the surgical environment from the camera tracking system in response to the robotic arm stopping its movement along the path.

At block 2052, processor 914 determines an estimated accuracy of the indication of the current position of the objects. In some embodiments, the objects can be moving within the surgical environment. Increased mobility of the objects can reduce the accuracy of their position based on a need to constantly be updating the position information. In additional or alternative examples, as an object (or more specifically its corresponding reference marker) gets closer to the camera tracking system, the camera tracking system can more precisely determine its position. Similarly, as an object gets farther from the camera tracking system the precision of its position can go down. In additional or alternative examples, objects can pass behind or under other objects reducing an ability of the camera tracking system from accurately determining a location of the object.

At block 2060, processor 914 receives an indication of a current position of the robotic arm from the camera tracking system. In some embodiments, the operation to receive the indication of the current position of the robotic arm from the camera tracking system includes at least one of: periodically receive the indication of the current position of the robotic arm from the camera tracking system; receive the indication of the current position of the robotic arm from the camera tracking system in response to detecting a collision; and receive the indication of the current position of the robotic arm from the camera tracking system in response to the robotic arm stopping its movement along the path.

At block 2062, processor 914 determines an estimated accuracy of the indication of the current position of robotic arm. In some embodiments, the robotic arm's movement can reduce the accuracy of the position information reported from the camera tracking system based on a need to constantly be updating the position information. In additional or alternative examples, as the robotic arm (or more specifically its corresponding reference marker) gets closer to the camera tracking system, the camera tracking system can more precisely determine its position. Similarly, as a robotic arm gets farther from the camera tracking system the precision of its position can go down. In additional or alternative examples, objects can pass behind or under other objects reducing an ability of the camera tracking system from accurately determining a location of the robotic arm.

At block 2070, processor 914 determines an expected current position of the objects path. At block 2080, processor 914 determines an expected current position of the robotic arm based on the path and speed of the robotic arm. In some embodiments, a precision/accuracy of a camera tracking system for determining a position of an object or the robotic arm may become poor (e.g., because the object or robotic arm moves behind or under another objects reducing or eliminating an ability of the camera tracking system from accurately determining a location of the object). In some examples, the surgical robot system can estimate an expected current position of the object and/or the robotic arm based on its prior location and speed (and in the case of the robotic arm its intended path).

At block 2090, processor 914 updates the path for the robotic arm based on the current position of the robotic arm and the current position of the objects (e.g., based on the indication of the current position of each object, the estimated accuracy of the indication of the current position of the objects, the indication of a current position of the robotic arm, the estimated accuracy of the indication of the current position of robotic arm, the expected current position of the objects path, and/or the expected current position of the robotic arm based on the path and speed of the robotic arm). In some embodiments, the operation to update the path for robotic arm includes to: determine a mobility of an object of the second plurality of objects; determine a proximity threshold associated with the object based on the mobility of the object; and update the path to avoid the path from being within the proximity threshold of the object. In some embodiments, depending on the estimated accuracy of the positioning information from the camera tracking system the expected current position of certain objects and/or the robotic arm can be given more or less weight. In additional or alternative embodiments, a proximity/collision tolerance of objects can be adjusted based on the estimated accuracy of the positioning information from the camera tracking system.

Various operations from the flow chart of FIG. 20 may be optional with respect to some embodiments of surgical robot systems and related methods.

### Further Definitions and Embodiments

In the above-description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

When an element is referred to as being “connected”, “coupled”, “responsive”, or variants thereof to another element, it can be directly connected, coupled, or responsive to the other element or intervening elements may be present. In contrast, when an element is referred to as being “directly connected”, “directly coupled”, “directly responsive”, or variants thereof to another element, there are no intervening elements present. Like numbers refer to like elements throughout. Furthermore, “coupled”, “connected”, “responsive”, or variants thereof as used herein may include wirelessly coupled, connected, or responsive. As used herein, the singular forms “a”, “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of present inventive concepts. The same reference numerals or the same reference designators denote the same or similar elements throughout the specification.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item. The common abbreviation “i.e.”, which derives from the Latin phrase “id est,” may be used to specify a particular item from a more general recitation.

Example embodiments are described herein with reference to block diagrams and/or flowchart illustrations of computer-implemented methods, apparatus (systems and/or devices) and/or computer program products. It is understood that a block of the block diagrams and/or flowchart illustrations, and combinations of blocks in the block diagrams and/or flowchart illustrations, can be implemented by computer program instructions that are performed by one or more computer circuits. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and/or flowchart block or blocks, and thereby create means (functionality) and/or structure for implementing the functions/acts specified in the block diagrams and/or flowchart block(s).

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and/or flowchart block or blocks. Accordingly, embodiments of present inventive concepts may be embodied in hardware and/or in software (including firmware, resident software, micro-code, etc.) that runs on a processor such as a digital signal processor, which may collectively be referred to as “circuitry,” “a module” or variants thereof.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Finally, other blocks may be added/inserted between the blocks that are illustrated, and/or blocks/operations may be omitted without departing from the scope of inventive concepts. Moreover, although some of the diagrams include arrows on communication paths to show a primary direction of communication, it is to be understood that communication may occur in the opposite direction to the depicted arrows.

Many variations and modifications can be made to the embodiments without substantially departing from the principles of the present inventive concepts. All such variations and modifications are intended to be included herein within the scope of present inventive concepts. Accordingly, the above disclosed subject matter is to be considered illustrative, and not restrictive, and the appended examples of embodiments are intended to cover all such modifications, enhancements, and other embodiments, which fall within the spirit and scope of present inventive concepts. Thus, to the maximum extent allowed by law, the scope of present inventive concepts is to be determined by the broadest permissible interpretation of the present disclosure including the following examples of embodiments and their equivalents, and shall not be restricted or limited by the foregoing detailed description.

What is claimed is:

1. A surgical robot system comprising:
processing circuitry; and
memory coupled to the processing circuitry and having instructions stored therein that are executable by the processing circuitry to cause the surgical robot system to perform operations to:
determine a plurality of actions to be completed by a surgical robot station during a surgery;
determine a plurality of potential positions in an operating room that the surgical robot station can be positioned during the surgery; and
generate a score associated with a potential position of the plurality of potential positions based on estimated movement of the surgical robot station required to perform the plurality of actions during the surgery from the potential position.

2. The surgical robot system of claim 1, wherein the surgical robot system comprises:
the surgical robot station, which includes:
a base configured to stabilize the surgical robot station at a desired position in the operating room; and
a robotic arm configured to hold a surgical tool at a plurality of different poses within a range of the robotic arm, each pose of the plurality of different poses having a different pose,
wherein the plurality of actions to be completed by the surgical robot station includes positioning the surgical tool at a plurality of poses relative to a patient, and
wherein the operation to generate the score includes to determine the score based on at least one of:
an estimated ability of the robotic arm to position the surgical tool at each pose of the plurality of poses relative to the patient;
an estimated amount of movement of the robotic arm to position the surgical tool at each pose of the plurality of poses relative to the patient;
an estimated type of movement of the robotic arm to position the surgical tool at each pose of the plurality of poses relative to the patient;
an estimated stability of the robotic arm while holding the surgical tool at each pose of the plurality of poses relative to the patient; and
an estimated angle of a joint associated with the robotic arm used to position the surgical tool at each pose of the plurality of poses relative to the patient.

3. The surgical robot system of claim 1, wherein each potential position of the plurality of positions includes a potential location and a potential orientation of the surgical robot station in the operating room.

4. The surgical robot system of claim 1, wherein the operation to generate the score comprises to quantify characteristics of simulated movement of the robotic arm performing the plurality of actions while the surgical robot station is positioned at the potential position.

5. The surgical robot system of claim 1, wherein the operation to generate the score includes to determine a plurality of scores that are each associated with one potential position of the plurality of potential positions.

6. The surgical robot system of claim 5, the operations further comprising to:
determine that the potential position of the plurality of potential positions is a desired position of the surgical robot system based on the score; and
output an indication of the desired position.

7. The surgical robot system of claim 6, wherein the operation to determine that the potential position is the desired position of the surgical robot station includes determining that the potential position is a center of gravity of a surface defined by the plurality of scores.

8. The surgical robot system of claim 7, wherein the operation to output the indication of the desired position includes to:
generate a heat map from the surface defined by the plurality of scores; and
display the heat map with a graphical element indicating the desired position.

9. The surgical robot system of claim 6, wherein the operation to output the indication of the desired position includes to:
determine a current position of the surgical robot station; and
output an indication of the current position of the surgical tool relative to the desired position of the surgical tool.

10. The surgical robot system of claim 9, wherein the operation to determine the current position of the surgical robot station includes to receive an indication of the current position from a camera tracking system.

11. The surgical robot system of claim 9, wherein the operation to output the indication of the current position of the surgical robot station includes at least one of:
display a virtual map including a virtual element representing the current position of the surgical tool and a virtual element representing the desired position of the surgical tool;
output an indication that the surgical tool is positioned at the desired position;
output an indication of a direction from the current position to the desired position;
output an indication of a distance between the current position and the desired position; and
transmit instructions to cause the surgical tool to move to the desired position.

12. The surgical robot system of claim 1, wherein the score is a first score,
wherein the potential position is a first potential position,
the operations further comprising to:
determine a second score associated with an ability of the surgical tool to perform a first portion of the plurality of actions at a second potential position of the plurality of potential positions and an ability of the surgical tool to perform a second portion of the plurality of actions at a third potential position of the plurality of potential positions; and
determine whether to split the operation into a plurality of segments based on the first score and/or the second score, each segment of the plurality of segments being associated with a different portion of the plurality of actions to be completed by the surgical tool.

13. The surgical robot system of claim 12, wherein the operation to determine whether to split the operation into the plurality of segments includes to determine to split the operation into the plurality of segments,
the operations further comprising to:
prior to the first segment, determine a first current position of the surgical tool;
prior to the first segment, output an indication of the first current position of the surgical tool relative to the second potential position of the surgical tool;
subsequent to the first segment and prior to the second segment, determine a second current position of the surgical tool; and
subsequent to the first segment and prior to the second segment, output an indication of the second current position of the surgical tool relative to the third potential position of the surgical tool.

14. The surgical robot system of claim 1, wherein the surgical robot system is configured to provide guidance to a surgeon performing a total knee arthroplasty (“TKA”).

15. A non-transitory computer readable medium having instructions stored therein that are executable by processing circuity of a surgical robot system to cause the surgical robot system to perform operations to:
determine a plurality of actions to be completed by a surgical robot station during a surgery;
determine a plurality of potential positions in an operating room that the surgical robot station can be positioned during the surgery;
generate a plurality of scores, each score associated with a corresponding potential position of the plurality of potential positions, based on estimated movement of the surgical robot station required to perform the plurality of actions during the surgery from the potential position;
determine that a first potential position of the plurality of potential positions is a desired position of the surgical robot system based on a first score of the plurality of scores that is associated with the first potential position; and
output an indication of the desired position.

16. The non-transitory computer readable medium of claim 15, wherein the surgical robot station includes:
a base configured to stabilize the surgical robot station at a desired position in the operating room; and
a robotic arm configured to hold a surgical tool at a plurality of different poses within a range of the robotic arm, each pose of the plurality of different poses having a different pose,
wherein the plurality of actions to be completed by the surgical robot station includes positioning the surgical tool at a plurality of poses relative to a patient, and
wherein the operation to generate the score includes to determine the score based on at least one of:
an estimated ability of the robotic arm to position the surgical tool at each pose of the plurality of poses relative to the patient;
an estimated amount of movement of the robotic arm to position the surgical tool at each pose of the plurality of poses relative to the patient;
an estimated type of movement of the robotic arm to position the surgical tool at each pose of the plurality of poses relative to the patient;
an estimated stability of the robotic arm while holding the surgical tool at each pose of the plurality of poses relative to the patient; and
an estimated angle of a joint associated with the robotic arm used to position the surgical tool at each pose of the plurality of poses relative to the patient.

17. The non-transitory computer readable medium of claim 15, wherein the operation to output the indication of the desired position includes causing the surgical robot system to:
determine a current position of the surgical robot station; and
output an indication of the current position of the surgical tool relative to the desired position of the surgical tool.

18. The non-transitory computer readable medium of claim 15, wherein the surgical robot system is configured to provide guidance to a surgeon performing a total knee arthroplasty (“TKA”).

19. A method of operating a surgical robot system, the method comprising:
determining a plurality of actions to be completed by a surgical robot station during a surgery;
determining a plurality of potential positions in an operating room that the surgical robot station can be positioned during the surgery; and
generating a score associated with a potential position of the plurality of potential positions based on estimated movement of the surgical robot station required to perform the plurality of actions during the surgery from the potential position.

20. The method of claim 19, wherein the surgical robot station includes:
a base configured to stabilize the surgical robot station at a desired position in the operating room; and
a robotic arm configured to hold a surgical tool at a plurality of different poses within a range of the robotic arm, each pose of the plurality of different poses having a different pose,
wherein the plurality of actions to be completed by the surgical robot station includes positioning the surgical tool at a plurality of poses relative to a patient, and
wherein determining the score includes determining the score based on at least one of:
an estimated ability of the robotic arm to position the surgical tool at each pose of the plurality of poses relative to the patient;
an estimated amount of movement of the robotic arm to position the surgical tool at each pose of the plurality of poses relative to the patient;
an estimated type of movement of the robotic arm to position the surgical tool at each pose of the plurality of poses relative to the patient;
an estimated stability of the robotic arm while holding the surgical tool at each pose of the plurality of poses relative to the patient; and
an estimated angle of a joint associated with the robotic arm used to position the surgical tool at each pose of the plurality of poses relative to the patient.
